(19) United States
(12) Patent Application Publication
(10) Pub. No.: US 2010/0210575 A1
(43) Pub. Date: Aug. 19, 2010

(54) STRUCTURING EFFECT OF CHOLESTEROL IN PEG-PHOSPHOLIPID MICELLES, DRUG DELIVERY OF AMPHOTERICIN B, AND COMBINATION ANTIFUNGALS

(75) Inventors: Glen S. Kwon, Waunakee, WI (US); Ronak J. Vakil, Springfield, NJ (US)

Correspondence Address: GREENLEE WINNER AND SULLIVAN P C, 4875 PEARL EAST CIRCLE, SUITE 200, BOULDER, CO, 80301, US

(73) Assignee: WISCONSIN ALUMNI RESEARCH FOUNDATION, Madison, WI (US)

(21) Appl. No.: 12/667,076

(22) PCT Filed: Jun. 27, 2008
(86) PCT No.: PCT/US08/68604

§ 371 Date: May 3, 2010

Related U.S. Application Data
(60) Provisional application No. 60/947,195, filed on Jun. 29, 2007.

(51) Int. Cl. A61K 31/7048; A01N 43/16; A61K 31/436; A61P 31/00; A01P 3/00

(52) U.S. Cl. 514/31; 514/291

(57) ABSTRACT

The disclosure herein relates to embodiments of compositions and methods in connection with polymeric micelles including PEG-phospholipids. Embodiments also relate to the controlled release of pharmaceutical agents in the context of drug delivery. Further disclosed are embodiments of PEG-DSPE/Cholesterol micelle formulations prepared with an antifungal agent, Amphotericin B, with capabilities including slow release of the agent in a deaggregated state. In embodiments, micellar preparations with Amphotericin B are compatible with solubility in aqueous salt solutions, thus allowing for concurrent co-administration of other pharmaceutical agents and/or sodium supplementation. In embodiments, polymeric micelle compositions are employed in combination antifungal therapeutic approaches such as Amphotericin B and other antifungal agents. Also disclosed herein are compositions and methods relating to combinations including AmB:PEG-DSPE, rapamycin:PEG-DSPE, and/or 5-fluorocytosine.

### CROSS-REFERENCES TO RELATED APPLICATIONS

This application claims the benefit of U.S. Provisional Application No. 60/947,195, filed Jun. 29, 2007, which is incorporated by reference to the extent not inconsistent herewith.

### STATEMENT ON FEDERALLY SPONSORED RESEARCH OR DEVELOPMENT

This invention was made with United States government support awarded by the following agency: NIH AI043346. The United States has certain rights in the invention.

### BACKGROUND OF THE INVENTION

Amphotericin B (AmB) remains at the “gold standard” level as the drug of choice with respect to clinically available antifungal agents, in part because of its potency and broad spectrum of activity. For example, AmB is widely considered effective for invasive candidiasis despite the facts that it has a profile of being poorly water soluble in addition to having difficulties in connection with both formulation and administration. Although it is generally insoluble in water, it can be solubilized by sodium deoxycholate. The classic amphotericin B deoxycholate (Fungizone™) formulation has been available since about 1960 and is a colloidal suspension of amphotericin B. A major disadvantage of sodium deoxycholate formulations of AmB (D-AmB), however, is toxicity. D-AmB is frequently associated with severe systemic adverse effects, including nephrotoxicity and hepatotoxicity.

Newer amphotericin preparations have been formulated combining amphotericin with lipid structures for intravenous administration. Three lipid preparations of amphotericin B have also been developed: (a) Amphotericin B Colloidal Dispersion (ABCD; Amphocil™ or Amphotec™); (b) Amphotericin B Lipid Complex (ABLC; Abelcet™); and (c) Liposomal Amphotericin B (L-AMB; Ambisome™). Despite this further development of antifungal agents, Fungizone remains one of the most effective agents in the treatment of systemic fungal infections.

Thus problems with currently existing formulations of important pharmaceutical agents, including antifungal agents, relate to less than satisfactory aspects of toxicity, drug delivery parameters (including, e.g., controlled release and/or aggregation state), efficacy, compatibility with clinical intravenous fluids, and deficiencies for options of combination antifungal compositions and methods. Therefore there is a recognized need in the art for new approaches in the field of pharmaceutical agents. The disclosure and embodiments of the present invention provide novel, alternative, and/or improved compositions and methods which can address one or more of the current problems and provide important advances in the state of the art.

### SUMMARY OF THE INVENTION

In general the terms and phrases used herein have their art-recognized meaning, which can be found by reference to standard texts, journal references and contexts known to those skilled in the art.

The following abbreviations are applicable. Amb, Amphotericin B; D-AmB, AmB solubilized by sodium deoxycholate (also referred to as desoxycholate); BSA, bovine serum albumin; PEG, polyethylene glycol; DSPE, 1,2-Distearoyl-sn-Glycero-3-Phosphoethanolamine; PEG-DSPE, 1,2-Distearoyl-sn-Glycero-3-Phosphoethanolamine-N-Methoxy(Polyethylene glycol); 5-FC, 5-fluorocytosine; SEC, size exclusion chromatography; DLS, Dynamic Light Scattering; PC, phosphatidylcholine.

In an embodiment, the invention provides a composition of PEG-PL:STX:RX for delivery of a pharmaceutical agent, comprising a poly(ethylene glycol)-phospholipid (PEG-PL), a sterol (STX), and the pharmaceutical agent (RX), wherein said RX is provided in a deaggregated and substantially micellar phase. In an embodiment, said phospholipid is 1,2-Distearoyl-sn-glycero-3-phosphoethanolamine (DSPE) and thereby said PEG-phospholipid is PEG-DSPE, thus PEG-DSPE:STX:RX. In an embodiment, said sterol is cholesterol (CHOL), thus PEG-DSPE:CHOL:RX.

In an embodiment, the pharmaceutical agent is a polyene antibiotic. In an embodiment, the pharmaceutical agent is Amphotericin B (AmB). In an embodiment, the pharmaceutical agent is selected from the group consisting of Amphotericin B (AmB) and rapamycin. In an embodiment, the pharmaceutical agent is a rapamycin analog, preferably for some applications a rapamycin analog having reduced immunosuppressive activity. In a specific embodiment, for example, the pharmaceutical agent is selected from the group consisting of: (S)—NOHCON-Piperidyl-Rapamycin; 1,2,3,4-tetrahydro-rapamycin; (S)—NOHCOOiBu-Rapamycin; (S)-2-Me-Thienyl-Rapamycin; (S)—OH-Rapamycin; and Desmethoxyrapamycin. In an embodiment, the pharmaceutical agent is a rapamycin prodrug. In a specific embodiment, for example, the pharmaceutical agent is selected from the group consisting of: 42-[3-Hydroxy-2-(hydroxymethyl)-2-methylpropanoate]rapamycin (Temsirolimus/CCI-779); 42-O-(2-Hydroxy)ethyl rapamycin (Everolimus/RAD001); Deforolimus/MK-8669; 42-(Dimethylphosphinate)rapamycin; Mono-(28)-N,N-dimethylglycinate-rapamycin; Mono-(28)-4-(pyrrolidino)butyrate-rapamycin; and Mono-(28)-N,N-diethylpropionate rapamycin.

In an embodiment, the phospholipid is DSPE, said sterol is cholesterol (CHOL), and said pharmaceutical agent is Amphotericin B; thus PEG-DSPE:CHOL:AmB.

In an embodiment, the micellar phase comprises micelles having an average diameter of less than about 100 nanometers. In an embodiment, the micellar phase comprises micelles having an average diameter of less than about 100 nanometers and greater than about 10 nanometers.

In an embodiment, the sterol is selected from the group consisting of: cholesterol, ergosterol, lanosterol, p-sitosterol or stigmasterol.

In an embodiment, the pharmaceutical agent is hydrophobic.

In an embodiment, a composition of the invention further comprises an aqueous salt solution. In an embodiment, said aqueous salt solution comprises aqueous sodium chloride (NaCl).

In an embodiment, the invention provides a composition of particles comprising AmB, PEG-DSPE, and CHOL having a ratio of from about 1:2:0.25 to about 1:2:2 for AmB:PEG-DSPE:CHOL.

In an embodiment, the invention provides a pharmaceutical formulation of a pharmaceutical agent, comprising a composition of the invention and a pharmaceutical carrier and/or excipient.

In an embodiment, the invention provides a method for delivery of a pharmaceutical agent to a subject, comprising providing a composition or formulation of the invention, and administering said composition or formulation to said subject, thereby achieving delivery of said pharmaceutical agent to said subject. In an embodiment of the method, a sterol is capable of reducing a first release rate of said pharmaceutical agent in said PEG-PL:STX:RX composition relative to a second release rate of the pharmaceutical agent in a PEG-PL:RX composition. In an embodiment, said first release rate and said second release rate are independently measured in a proteinaceous fluid.

In an embodiment, the invention provides a combination antifungal composition comprising a composition as described herein and a second pharmaceutical agent, wherein at least one of said pharmaceutical agent and said second pharmaceutical agent is an antifungal agent. In an embodiment, an antifungal agent is Amphotericin B. In an embodiment, an antifungal agent is fluconazole. In an embodiment, an antifungal agent is 5-fluorocytosine. In an embodiment, a pharmaceutical agent is Amphotericin B and a second pharmaceutical agent is fluconazole or 5-fluorocytosine. In an embodiment, at least one of a first pharmaceutical agent and a second pharmaceutical agent is selected from the group consisting of Amphotericin B, rapamycin, nystatin, 5-fluorocytosine and fluconazole (also known as 2-(2,4-difluorophenyl)-1,3-bis(1H-1,2,4-triazol-1-yl)propan-2-ol). In an embodiment, at least one of a first pharmaceutical agent and a second pharmaceutical agent is a rapamycin analog, preferably for some applications a rapamycin analog having reduced immunosuppressive activity. In a specific embodiment, for example, at least one of a first pharmaceutical agent and a second pharmaceutical agent is selected from the group consisting of: (S)—NOHCON-Piperidyl-Rapamycin; 1,2,3,4-tetrahydro-rapamycin; (S)—NOHCOOiBu-Rapamycin; (S)-2-Me-Thienyl-Rapamycin; (S)—OH-Rapamycin; and Desmethoxyrapamycin. In an embodiment, at least one of a first pharmaceutical agent and a second pharmaceutical agent is a rapamycin prodrug. In a specific embodiment, for example, at least one of a first pharmaceutical agent and a second pharmaceutical agent is selected from the group consisting of: 42-[3-Hydroxy-2-(hydroxymethyl)-2-methylpropanoate]rapamycin (Temsirolimus/CCI-779); 42-O-(2-Hydroxy)ethyl rapamycin (Everolimus/RAD001); Deforolimus/MK-8669; 42-(Dimethylphosphinate)rapamycin; Mono-(28)-N,N-dimethylglycinate-rapamycin; Mono-(28)-4-(pyrrolidino)butyrate-rapamycin; and Mono-(28)-N,N-diethylpropionate rapamycin.

In an embodiment, the invention provides a method of co-administration of a plurality of pharmaceutical agents to a subject in need thereof, comprising:

(a) providing a micelle composition of a first pharmaceutical agent according to a composition of the invention as described herein, wherein said micelle composition is compatible with an aqueous NaCl solution for sodium supplementation to said subject;
(b) providing at least a second pharmaceutical agent;
(c) mixing said first and second pharmaceutical agents, thereby generating a mixture; and
(d) administering said mixture to said subject; thereby achieving co-administration of said plurality of pharmaceutical agents.

In an embodiment, the method further comprises a step (e) administering a sodium supplementation to said subject, wherein the administering of said sodium is before, concurrent with, or after step (d). In an embodiment, at least one of said plurality of pharmaceutical agents is an antifungal agent. In an embodiment, said first pharmaceutical agent comprises Amphotericin B. In an embodiment, each of said first and second pharmaceutical agents is an antifungal agent. In an embodiment, said second pharmaceutical agent is rapamycin, 5-fluorocytosine, or fluconazole.

In an embodiment, the invention provides a method of making an AmB composition, wherein said AmB composition is in a substantially deaggregated form and capable of being soluble in an aqueous salt solution, comprising providing AmB, providing PEG-DSPE, and mixing said AmB with said PEG-DSPE; thereby generating said AmB composition.

In an embodiment, the invention provides a method of making an AmB composition, wherein said AmB composition is in a substantially deaggregated form and capable of being soluble in an aqueous salt solution, comprising providing AmB, providing PEG-DSPE, providing CHOL, dissolving said AmB, PEG-DSPE, and CHOL in a solvent mixture comprising methanol and chloroform, evaporating said solvent to allow dissolution of a thin film sample, equilibrating said sample at a temperature of 50 degrees centigrade, and filtering said sample using an 0.45-micron filter of polyethersulfone; thereby generating said AmB composition. In an embodiment, it is not necessary to formulate said AmB composition with sodium deoxycholate.

In an embodiment, the invention provides a method of preparing a composition of the invention as described herein. In an embodiment, the invention provides a method of preparing a pharmaceutical formulation as described herein.

In an embodiment, the invention provides a micelle composition of PEG-PL:RX comprising a poly(ethylene glycol)-phospholipid (PEG-PL), and the pharmaceutical agent (RX). In an embodiment, PEG-PL:RX is PEG-DSPE:RX. In an embodiment, the invention provides a micelle composition of PEG-PL:STX:RX, comprising a poly(ethylene glycol)-phospholipid (PEG-PL), a sterol (STX), and a pharmaceutical agent (RX).

In an embodiment, the invention provides a combination antifungal composition comprising an Amphotericin B component and at least a second antifungal agent, wherein said Amphotericin B and said second antifungal agent are solubilized in an aqueous salt solution. In an embodiment, the Amphotericin B component comprises AmB:PEG-DSPE. In an embodiment, the second antifungal agent comprises a rapamycin component. In an embodiment, the rapamycin component comprises rapamycin:PEG-DSPE. In an embodiment, the second antifungal agent comprises 5-fluorocytosine.

In an embodiment, the combination antifungal composition further comprises a third antifungal agent. In an embodiment of a combination antifungal composition, said Amphotericin B component comprises AmB:PEG-DSPE, said second antifungal agent comprises rapamycin:PEG-DSPE, and said third antifungal agent comprises 5-fluorocytosine.

In an embodiment, the invention provides a method of inhibiting a fungal agent, comprising: contacting said fungal agent with a composition as described herein. In an embodiment, the composition is a combination antifungal composition. In an embodiment, the invention provides a method of treating a fungal infection, comprising administering a composition of the invention to a subject in need thereof, thereby treating the fungal infection.

In an embodiment, the invention provides compositions and methods for the slow release of a pharmaceutical agent. In an embodiment, the invention provides compositions and methods for maintaining a pharmaceutical agent in a deaggregated state. In an embodiment, the pharmaceutical agent is an antifungal agent. In an embodiment, the pharmaceutical agent is Amphotericin B. In an embodiment, the slow release of AmB can reduce AmB-related toxicity in a host subject.

In an embodiment, the invention provides compositions and method in connection with a delivery system for AmB. In an embodiment, the delivery system comprises PEG-DSPE micelles into which cholesterol has been incorporated. In an embodiment, the cholesterol can have a structuring effect on the micelles. In an embodiment, the micelles can facilitate solubilization of AmB. In an embodiment, the solubilization is in an aqueous salt solution. In an embodiment, the micelles are stable in the presence of NaCl. In an embodiment, compositions and methods of the invention provide an advantage in aiding in the protection against the dose-limiting kidney toxicity associated with AmB.

In an embodiment, a composition of the invention comprises a PEG-phospholipid component, a cholesterol component, and a pharmaceutical agent. In embodiments of the foregoing, the composition does not comprise a further lipid or phospholipid component. In a particular embodiment, a composition comprises PEG-DSPE:CHOL but does not further comprise another lipid component or phospholipid component. In another particular embodiment, a composition comprises PEG-DSPE:CHOL but does not further comprise DSPC.

In an embodiment, a composition of the invention consists essentially of a PEG-phospholipid component, a cholesterol component, and a pharmaceutical agent.

In an embodiment, PEG-DSPE micelles incorporating cholesterol demonstrate decreased mobility in the micelle core. This mobility observation can be due to hydrophobic interactions with the rigid sterol. AmB is readily incorporated in these novel micelles. In contrast with PEG-DSPE micelles, the micelles with the cholesterol component can slowly release the AmB in the presence of serum albumin. Since albumin is the most abundant protein in serum, this result indicates that an embodiment of the invention can achieve slow release of deaggregated AmB. Such slow release can result in advantages, e.g., lowered host toxicity without loss of potent activity of AmB, an important antifungal drug.

In an embodiment, a composition of the invention has a noncrystalline form. In an embodiment, a composition of the invention has a liquid ordered phase.

In an embodiment, a composition of the invention is isolated or purified.

In an embodiment, the invention provides a method of controlling a fungal agent, comprising contacting the fungal agent with a composition of the invention. In an embodiment, the controlling is by inhibiting. In an embodiment, the fungal agent is contacted in vitro. In an embodiment, the fungal agent is contacted in vivo. In an embodiment, the fungal agent is contacted in vivo in a mammal. In an embodiment, the fungal agent is contacted in a human.

In an embodiment, a composition or method of the invention is useful in the treatment of candidiasis, cryptococcoses, aspergillosis, histoplasmosis, blastomycosis, coccidioidomycosis, as well as other fungi. In an embodiment, a fungal agent is Candida, Cryptococcus neoformans, Aspergillus, Histoplasma, Blastomyces, Coccidiodes, or other fungi.

In embodiments of the invention, compositions and/or methods are compatible with sodium supplementation to a subject. In an embodiment, the sodium supplementation is administered before, concurrent with, or after administration of a composition of the invention. In an embodiment, sodium supplementation is used in part as a protective measure for the kidneys. In a particular embodiment, clinically, sodium supplementation is done before or after the infusion of a composition comprising amphotericin B, avoiding or reducing precipitation of the drug, solubilized by sodium deoxycholate.

In an embodiment, the invention provides a pharmaceutical formulation comprising a composition of the invention. In an embodiment, the invention provides a method of synthesizing a composition of the invention or a pharmaceutical formulation thereof. In an embodiment, a pharmaceutical formulation comprises one or more excipients, carriers, and/or other components as would be understood in the art. In an embodiment, an effective amount of a composition of the invention can be a therapeutically effective amount.

In an embodiment, the invention provides a method for treating a medical condition comprising administering to a subject in need thereof, a therapeutically effective amount of a composition of the invention. In an embodiment, the medical condition is a fungal infection. In an embodiment, the condition is a systemic fungal infection.

In an embodiment, the invention provides a medicament which comprises a therapeutically effective amount of one or more compositions of the invention. In an embodiment, the invention provides a method for making a medicament for treatment of a condition described herein.

Without wishing to be bound by any particular theory, there can be discussion herein of beliefs or understandings of underlying principles or mechanisms relating to embodiments of the invention. It is recognized that regardless of the ultimate correctness of any explanation or hypothesis, an embodiment of the invention can nonetheless be operative and useful.

### BRIEF DESCRIPTION OF THE FIGURES

FIG. 1 illustrates results of the effect of cholesterol content on particle size in nm (y-axis) of PEG-DSPE|Cholesterol micelles (cholesterol:PEG-DSPE ratio indicated as mol:mol; size according to volume-weighted NICOMP average).

FIG. 2 illustrates observations of I_{M}/I_{E} ratios (y-axis) for P3P fluorescence emission in PEG-DSPE|Cholesterol micelles, with emission at λ_{θx}=333 nm, n=3.

FIG. 3 illustrates ¹H NMR spectra for (A) PEG-DSPE micelles; and (B) PEG-DSPE|Cholesterol micelles with PEG-DSPE: cholesterol=1:1 and temperature at 25° C.

FIG. 4 illustrates absorption spectra of AmB solubilized by PEG-DSPE and PEG-DSPE|Cholesterol micelles.

FIG. 5 illustrates AmB release profiles at 25° C. in: (A) 5 mM HEPES; and (B) 4% BSA. The y-axis indicates the fraction of AmB remaining.

FIG. 6, in panels a-c, illustrates absorbance spectra of micelle encapsulated AmB in 4% BSA. (A) Unencapsulated AmB (in DMSO); (B) AmB in PEG-DSPE micelles (AmB: PEG-DSPE=1:2); (C) AmB in PEG-DSPE|Cholesterol micelles (AmB: PEG-DSPE: Cholesterol=1:2:2). FIG. 6, in panels d-f, illustrates absorbance at 412 nm as a function of incubation time corresponding to panels a-c. The AmB concentration was 10 μg/mL.

FIG. 7 illustrates chemical structures of AmB, PEG-DSPE, and cholesterol.

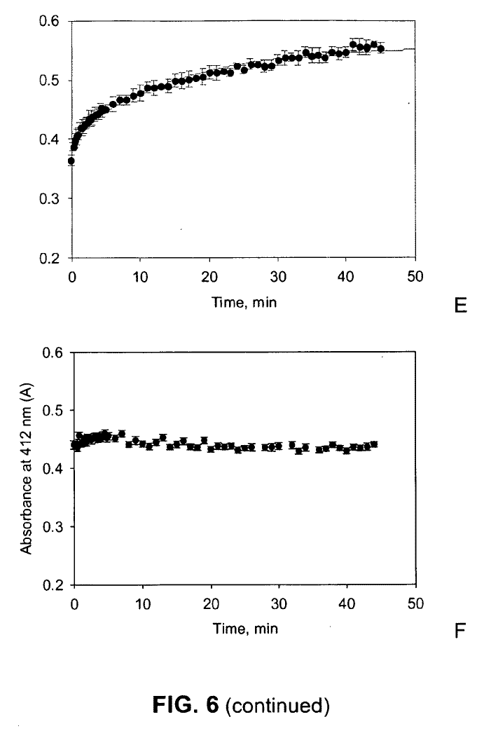

FIG. 8 illustrates an interaction and structuring effect of cholesterol in PEG-DSPE:cholesterol micelles.

FIG. 9 illustrates results of light scattering at 650 nm on admixing: a. conventional AmB (D-AmB) with 0.9% NaCl; b. D-AmB with 5% dextrose; c. AmB|PEG-DSPE with rapamycin/PEG-DSPE and 5-FC in 0.9% NaCl. The final AmB concentration was 0.1 mg/mL.

FIG. 10 illustrates results of SEC of AmB|PEG-DSPE, rapamycin|PEG-DSPE and 5-FC in 0.9% NaCl^{a,b} (where: ^{a}incubation at 37° C. for 4.5 h. ^{b} 40 Ξg/mL AmB|PEG-DSPE, 40 μg/mL rapamycin|PEG-DSPE, 2 mg/mL 5-FC). Column: Shodex PROTEIN-KW804. Eluent: Water at 0.75 mL/min. Column temperature: 10° C.

FIG. 11 illustrates results of sizing of AmB|PEG-DSPE, rapamycin|PEG-DSPE and 5-FC mixture in 0.9% NaCl using DLS. a. immediately after mixing b. after incubation for 12 h at room temperature.

FIG. 12. Contour Plots for a. 5-FC-AmB|PEG-DSPE b. 5-FC-rapamycin|PEG-DSPE c. rapamycin|PEG-DSPE-AmB|PEG-DSPE combinations against C. albicans 98-17.

FIG. 13. Contour Plots for 5-FC-AmB|PEG-DSPE-rapamycin|PEG-DSPE combinations for AmB|PEG-DSPE:rapamycin|PEG-DSPE a. 1:2 b. 2:1 for C. albicans 98-17.

FIG. 14. Contour Plots for a. 5-FC-AmB|PEG-DSPE b. 5-FC-rapamycin|PEG-DSPE c. rapamycin|PEG-DSPE-AmB|PEG-DSPE combinations against C. albicans 98-234.

FIG. 15. Contour Plots for 5-FC-AmB|PEG-DSPE-rapamycin|PEG-DSPE combinations for AmB|PEG-DSPE:rapamycin|PEG-DSPE a. 1:2 b. 2:1 for C. albicans 98-234.

### DETAILED DESCRIPTION OF THE INVENTION

When used herein, the term “micelle” can refer to a component or composition wherein a substantial portion of the material is in the form of micelles. In an embodiment in connection with drug delivery, certain compositions of particles formed from amphiphilic materials (e.g., polymers) in the size range of from about 10 nm to about 0.1 micrometers can be referred to as micelles.

The invention may be further understood by the following non-limiting examples.

### Example 1

### Cholesterol and PEG-Phospholipid Micelles

This example at least in part relates to the development of micellar compositions with cholesterol and PEG-phospholipid components. The compositions are readily adaptable for combining with a pharmaceutical agent such as Amphotericin B. In a particular aspect the example describes the effect of cholesterol on the release of Amphotericin B from PEG-phospholipid micelles. In an embodiment, the release is controlled according to compositions and methods of the invention. In an embodiment, the controlled release is slow relative to certain approaches in the art other than according to an embodiment of the invention.

Abstract. The effect of varying levels of cholesterol on the properties of PEG-DSPE micelles has been studied in this work. Steady state fluorescence and ¹H NMR measurements point to increased microviscosity and reduced segmental mobility in PEG-DSPE micelles with cholesterol (PEG-DSPE|Cholesterol). These observations are consistent with increased order in lipid bilayers incorporating cholesterol. We have taken this information and, upon directing attention to problems in the field of drug delivery, conceived and developed useful applications.

We have discovered that PEG-DSPE|Cholesterol micelles solubilize high levels of the poorly water-soluble antifungal, amphotericin B (AmB), in a predominantly deaggregated form.

We observed that AmB release from PEG-DSPE micelles is slow in a non-proteinaceous aqueous buffer solution, but remarkably rapid in the presence of bovine serum albumin (BSA), pointing to a destabilizing effect of this plasma protein on PEG-DSPE micelles. In contrast, AmB release from PEG-DSPE|Cholesterol micelles is slow in both buffer and BSA solutions. The differences in the absorption spectra of AmB encapsulated in PEG-DSPE and PEG-DSPE|Cholesterol micelles suggest that cholesterol incorporation has a marked effect on AmB incorporation in the micelle core. Electronic absorption studies further illustrate that in contrast with PEG-DSPE micelles, AmB is stably incorporated in PEG-DSPE|Cholesterol micelles and is released slowly, presumably by a diffusion-controlled process. We therefore demonstrate compositions and methods for controlled release which can be useful for physiologically relevant conditions of drug delivery.

Introduction. Amphotericin B (AmB) is a poorly soluble antifungal drug used to treat systemic fungal diseases, despite severe toxicities. AmB toxicity is thought to be mediated at least in part by the relative aggregation state of the drug. We have explored the utility of polymeric micelles to solubilize AmB and to reduce its toxicity without an appreciable loss in antifungal activity. We have recently described our efforts to study PEG-DSPE micelles for the delivery of AmB (reference 1). P EG-DSPE micelles could incorporate and deaggregate AmB on the basis of absorption spectroscopy. AmB encapsulated in PEG-DSPE micelles simultaneously exerted less hemolytic activity than free AmB and retained potent antifungal activity. This two-fold characteristic suggested that AmB is released from PEG-DSPE micelles in a monomeric form, which is selectively active against fungal cells (2-5). However, we found that the in vivo toxicity of this formulation correlated poorly with in vitro assays and was comparable to the standard formulation, D-AmB (AmB solubilized by sodium deoxycholate). For embodiments of compositions of the present invention, formulations are able to be used both in vitro and in vivo.

Research has recently shown that administration of D-AmB as a continuous infusion over 24 hours is better tolerated compared to an equal dose administered as a standard 2-4 hour infusion (6-10). It is becoming increasingly clear that slow delivery of AmB reduces toxic manifestations of this important antifungal drug. Hence, from the perspective of AmB delivery using polymeric micelles, we have directed efforts towards compositions and methods for achieving a slow release of the encapsulated drug in connection with acceptable and/or lower in vivo toxicity. We have investigated AmB release in aqueous buffer and in the presence of serum albumin using a dialysis setup. PEG-DSPE micelles release AmB slowly in buffer, presumably in a monomeric form. However, serum albumin appears to have a destabilizing effect on these micelles, leading to rapid dissociation of encapsulated AmB.

Research on phospholipid bilayers in a liquid-crystalline state indicates that cholesterol incorporation into phospholipid bilayers has a marked effect on the chain mobility and orientational order (11). We recognized the potential significance of this information and investigated the effect of cholesterol incorporation on the properties of PEG-DSPE micelles. Our drug-release studies indicate that in contrast with PEG-DSPE micelles, AmB release from PEG-DSPE micelles co-incorporating cholesterol (PEG-DSPE|Cholesterol) is slow in the presence of serum albumin. Steady-state fluorescence measurements and ¹H-NMR spectra correspond to decreased mobility in the core of PEG-DSPE|Cholesterol micelles. The absorbance spectrum of AmB in PEG-DSPE|Cholesterol micelles displays narrower peaks than in PEG-DSPE micelles—an effect perhaps related to increased rigidity in the micelle core. Results from AmB absorption kinetics in the presence of a physiological concentration of serum albumin are in agreement with drug-release studies and point to stable incorporation of AmB in PEG-DSPE|Cholesterol micelles. Because albumin is the most abundant serum protein, the in vivo release of AmB from PEG-DSPE|Cholesterol micelles can be advantageously slow, an outcome that can influence in vivo toxicity for this central antifungal agent.

Materials. AmB was obtained as a gift from Alpharma (Copenhagen, Denmark) and was stored at −20° C. until use. PEG-DSPE (Mₙ=5800 g/mol) was obtained from Avanti Polar Lipids (Alabaster, Ala.). Cholesterol was obtained from Aldrich (Milwaukee, Wis.). 1,3-(1,1′-dipyrenyl)propane (P3P) was obtained as a gift from Dr. M. Yokoyama (Kanagawa Academy of Science and Technology, Japan). All other reagents used were of analytical grade and were used without further purification.

Micelle preparation and incorporation of AmB. PEG-DSPE (6.0 mg/mL in chloroform), cholesterol (1 mg/mL in chloroform), and AmB (0.25 mg/mL in methanol) stock solutions were mixed in a round bottom flask to obtain the desired AmB: cholesterol:PEG-DSPE ratio. The organic solvent was evaporated under vacuum to produce a thin film of co-precipitated drug and polymer. This film was dissolved in 10 mM HEPES, pH 7.0 and incubated at 50° C. for 10 min to allow for complete equilibration. The micellar solution was filtered through a 0.45-μm polyethersulfone (PES) syringe-filter. Empty micelles were prepared by using an identical procedure without drug. The concentration of AmB was quantified by diluting a 50 μL aliquot of AmB in 1.95 mL DMF and by observing absorbance at 413.5 nm. This assay was tested for linearity in the 0.02-0.8 mg/mL range.

### Micelle Characterization.

Dynamic Light Scattering. Micelle diameters were determined by using the NICOMP ZLS380 particle sizer (Particle Sizing Systems, Santa Barbara, Calif.). Data was acquired to have at least 100 k counts in Channel 1. The light scattering data was interpreted by using Gaussian and NICOMP analyses. NICOMP analysis permits the deconvolution of light-scattering data into multimodal distributions. Particle sizes were expressed as volume-weighted diameters.

Core microviscosity measurements using 1,3-(1,1′-dipyrenyl)propane. Microviscosity was estimated by measuring the ratio of the fluorescence intensities of the monomer and the excimer; that is we measured the I_{M}/I_{E} of P3P emission at 376 and 480 nm respectively (12, 13). Aqueous solutions containing 0.5 mg/mL polymer were aliquoted into tubes containing P3P. The samples were heated to 65° C. for 1 hour and were allowed to equilibrate overnight at room temperature. The concentration of P3P in the aqueous solution was 0.2 μM. Samples were excited at 333 nm, and fluorescence emission at room temperature was recorded from 350 to 600 nm using a Fluoromax 3 fluorimeter (Horiba Jobin Yvon, Edison, N.J.). The spectra were analyzed using the Datamax software (version 2.20, Horiba Jobin Yvon, Edison, N.J.).

¹H NMR spectroscopy. Samples for ¹H-NMR were prepared by dissolving the co-precipitated film of PEG-DSPE and cholesterol in deuterium oxide (Aldrich, Milwaukee) allowing for equilibration at 50° C. for 10 min. The samples contained 5 mg/mL PEG-DSPE. ¹H-NMR spectra were acquired at 25° C. on a 400 MHz Varian spectrometer using the using the standard two pulse sequence (14). Peak line-widths (Δν_{1/2}) were determined using the Varian software (version 6.1 revision C, Palo Alto, Calif.).

AmB release from PEG-DSPE micelles using equilibrium dialysis. AmB stock solutions (micelle encapsulated or dissolved in DMSO) were diluted to 30 μg/mL in HEPES buffer or in 4% BSA. These solutions were put in dialysis cassettes (MWCO 7000 g/mol, Pierce) and were placed in excess buffer (5 mM HEPES, pH 7.0). The MWCO of the dialysis membrane was chosen to allow for diffusion of AmB (molecular weight 924.1) while retaining large molecules such as bovine serum albumin (molecular weight 66 kDa) and intact micelles (molecular weight >100 kDa). Near-sink conditions for AmB were maintained by performing the release experiment in a 100-fold excess dialysis buffer. Because AmB is susceptible to degradation, the dialysis buffer was degassed overnight and 20 μg/mL propyl gallate was added as an antioxidant (15, 16). At predetermined time intervals, 100 μL samples were taken and AmB concentration in dialysis cassettes was determined using reversed-phase HPLC. Protein from samples containing serum albumin was first precipitated by addition of 400 μL cold methanol. After centrifugation at 13,200 RPM (16.1×10³ g) for 10 min, the supernatant was extracted and analyzed for AmB content, using reversed-phase HPLC.

AmB release was fit to a first-order process of the form Aₜ/A₀=a.e^{-k.t} by non-linear regression (SigmaPlot v.9.0). Aₜ corresponds to AmB concentration in the dialysis cassette at time t and A₀ is the initial concentration. The half-life t_{1/2} for AmB release was calculated using t_{1/2}=0.693/k.

For determination of AmB content, 15 μL samples were injected into 4.6 mm×150 mm Eclipse XDB-C8 reversed-phase column (Agilent Technologies) and AmB absorbance detected at 412 nm using the diode array detector. The mobile phase consisted of a linear gradient of methanol—(5% v/v acetic acid). The column was maintained at 25° C. and the flow rate of mobile phase was 2 mL/min. The assay was tested for linearity in the 0.1-100 μg/mL range.

Electronic absorption kinetics. Stock solutions containing AmB (micelle encapsulated or dissolved in DMSO) were added to 4% BSA, buffered to pH 7.0 with 10 mM HEPES. The final concentration of AmB was 10 μg/mL. For AmB dissolved in DMSO, the final DMSO content was less than 0.2%. Absorbance spectra were recorded from 300 to 450 nm at room temperature by using the Cary 50 spectrophotometer (Varian, Palo Alto, Calif.). Sequential spectra were acquired at 15 second intervals for the first 5 min; and at 1 minute intervals for an additional 40 minutes. A scan for baseline absorbance was taken before addition of AmB and was subtracted from each spectrum. The change in absorbance at 412 nm, A_{≈}−Aₜ was fit to a first-order process of the form A_{∞}−Aₜ=a.e^{-k.t} using non-linear regression (SigmaPlot v.9.0).

### Results and Discussion

Micelle preparation and characterization. PEG-DSPE and PEG-DSPE|Cholesterol micelles were prepared by using a solvent-evaporation method. PEG-DSPE micelles with a narrow size distribution were obtained by dissolving the film of co-precipitated drug and polymer at 25° C., according to Vakil 2005. Samples incorporating cholesterol had a broad particle size distribution when prepared in a similar manner, resulting in turbid solutions with variable mean particle sizes. PEG-DSPE|Cholesterol micelles with a narrow particle distribution could be obtained when the polymer film was dissolved at 50° C., allowing 10 min for equilibration. This result was presumably due to greater mobility of lipid chains at this elevated temperature.

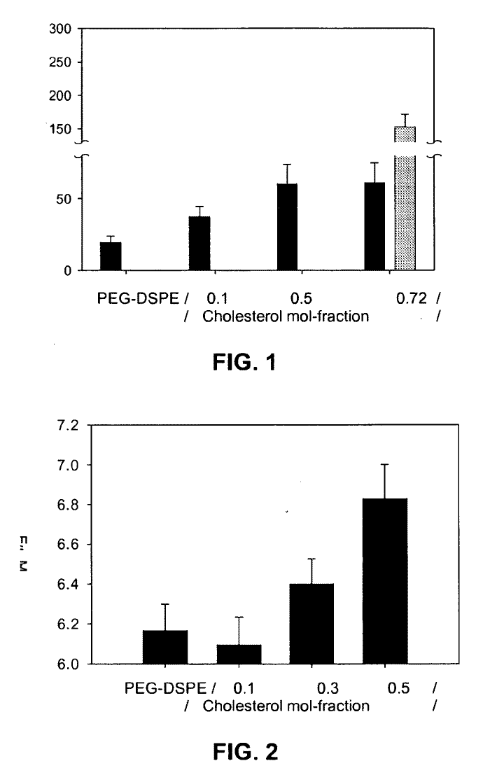

FIG. 1 illustrates the effect of cholesterol content on size of PEG-DSPE|Cholesterol micelles, with indicated cholesterol: PEG-DSPE ratios (mol:mol) and particle sizes indicated according to volume-weighted NICOMP average.

The mean diameter for empty PEG-DSPE micelles was ca. 16 nm, determined using dynamic light scattering. The diameter of PEG-DSPE|Cholesterol micelles increased in a composition-dependent manner and reached ca. 60 nm with a cholesterol mole-fraction of 0.5 (FIG. 1). For higher fractions of cholesterol incorporation, dynamic light scattering reported two populations—one that corresponded to PEG-DSPE|Cholesterol micelles (ca. 60 nm) and another to aggregates with sizes >150 nm.

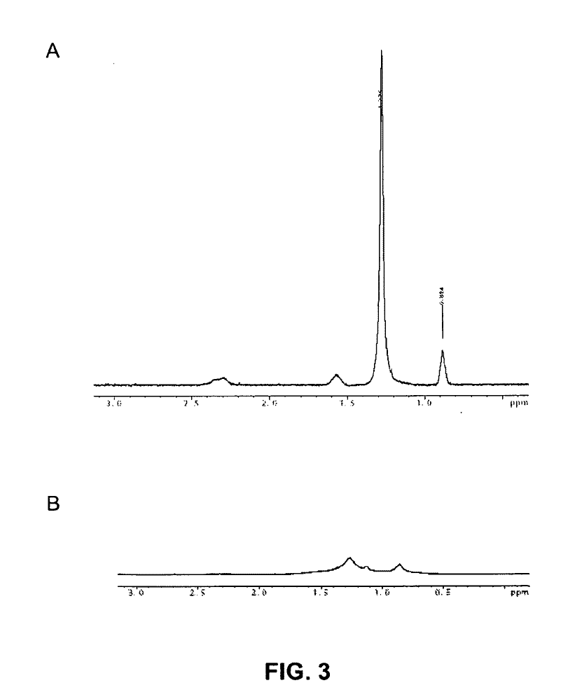

Intramolecular excimer formation of the lipophilic fluorescent probe P3P depends on the flexibility of the propylene chain between the pyrene moieties in the molecule. Because such conformational changes are restricted in environments of high viscosity, the I_{M}/I_{E} ratio is useful in determining the fluidity of the microenvironment sensed by these probes (12, 17). The I_{M}/I_{E} ratio increased with increasing cholesterol content from 6.2±0.13 in PEG-DSPE micelles to 6.8±0.17 (p<0.05) at 50% cholesterol incorporation (FIG. 2). The I_{M}/I_{E} ratio for P3P fluorescence in PEG-DSPE and PEG-DSPE|Cholesterol micelles was intermediate between the sodium dodecyl sulfate micelles (I_{M}/I_{E}˜2) with a liquid-like core and that of PEG-b-poly(c-caprolactone) micelles which have rigid, presumably partially crystalline cores (Iₘ/I_{E}˜22).

FIG. 2 shows results of I_{M}/I_{E} ratios for P3P fluorescence emission^{a} in PEG-DSPE|Cholesterol micelles (^{a}λₑₓ=333 nm ^{b}n=3).

The structuring effects of cholesterol in phospholipid vesicles have been reviewed by Ohvo-Rekila et al. (11) and McConnell et al. (18). FTIR studies on DPPC vesicles that contain cholesterol have shown that cholesterol is intercalated in the bilayer and that the rigid sterol head interacts with diacyl chains closer to the phospholipid head group. This hydrophobic interaction results in restricted trans-gauche isomerization in diacyl chains (19). The ordering of diacyl chains by cholesterol is not uniform through the DPPC bilayer; diacyl chains retain greater mobility away from the phospholipid head group (toward the centre of the bilayer) owing to interaction with the more flexible cholesterol side-chain. Molecular dynamics simulations performed by Hofsas et al. are consistent with observations from FTIR studies and further demonstrate that the average area per phospholipid molecule in the DPPC bilayer is significantly decreased on account of interaction with cholesterol (20). We have extended these observations to our interpretation of the ¹H-NMR results and of the P3P fluorescence results in PEG-DSPE|Cholesterol micelles. More importantly, in our hands this interpretation has translated into a significant advance where the effect of cholesterol in PEG-DSPE|Cholesterol micelles can serve to control release in the context of drug delivery.

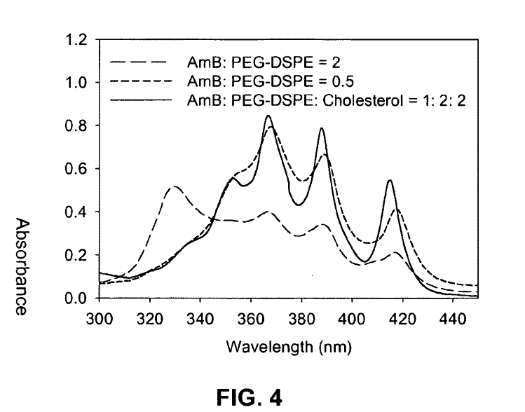

Restricted mobility in the core of polymeric micelles results in broadened line-widths in a ¹H-NMR spectrum (14, 21). FIG. 3 shows the ¹H-NMR spectrum of PEG-DSPE micelles in D₂O. High mobility in the core of PEG-DSPE micelles was inferred from the relatively sharp peaks corresponding to protons from the diacyl chains at 1.26 and 0.8 ppm. The width-at-half-height (Δν_{1/2}) for 1.26 and 0.8 ppm peaks were 8.9 Hz and 13.7 Hz, respectively. These results were consistent with electron spin resonance studies reported by Belsito et al. with 1,2-Dipalmitoyl-sn-glycero-3-phosphoethanolamine-N-methoxy(polyethylene glycol) (DPPE-PEG) micelles (22). Segmental mobility in PEG-lipid micelles is greater than comparable lipid bilayers, and Belsito et al. have attributed this phenomenon to decreased lipid packing induced by the bulky poly(ethylene glycol) head group (22-24). The incorporation of cholesterol in PEG-DSPE micelles resulted in a broadening of ¹H-NMR signals at 1.26 ppm (Δν_{1/2}˜55.3 Hz) and 0.88 ppm (Δν_{1/2}˜31.9 Hz), pointing to hindered motion of the diacyl chains in the micelle core. Greater mobility in the diacyl chain towards the micelle interior may be inferred for PEG-DSPE|Cholesterol micelles since the peak at 1.26 ppm corresponding to diacyl methylene protons is broader compared to the corresponding CH₃— protons at 0.8 ppm.

FIG. 3 illustrates ¹H NMR spectra for a. PEG-DSPE micelles and b. PEG-DSPE|Cholesterol micelles (PEG-DSPE: cholesterol=1:1) at 25° C.

AmB encapsulation and analysis of absorbance spectra of encapsulated drug. It is possible to incorporate high levels of AmB in PEG-DSPE micelles by using a solvent evaporation method (Table 1). The fraction of initial AmB encapsulated by the PEG-DSPE|Cholesterol micelles (84%) was similar to that for PEG-DSPE micelles (78%) using this method. The particle size of PEG-DSPE|Cholesterol micelles was dependant on the level of cholesterol incorporation and reached ca. 60 nm at 50% cholesterol (mol:mol). See Table 1.

TABLE 1
Solubilization of AmB by PEG-DSPE
and PEG-DSPE|Cholesterol micelles

| Molar ratio | Fraction of | AmB | | Hydrodynamic |
|---|---|---|---|---|
| AmB:PEG-DSPE: | initial AmB | loading | [AmB], | Diameter |
| Cholesterol^{a} | encapsulated | % w/w | mg/mL^{b} | (nm) |
| 1:0.5 | 0.78 | 24.2 | 0.39 ± 0.02 | 21.8 ± 6.1^{c} |
| 1:2 | 0.78 | 7.21 | 0.38 ± 0.12 | 19.3 ± 4.4^{c} |
| 1:2:0.25 | 0.82 | 7.53 | 0.41 ± 0.09 | 39.3 ± 4.7^{d} |
| 1:2:0.5 | 0.88 | 7.93 | 0.44 ± 0.12 | 64.6 ± 9.2^{d} |
| 1:2:2 | 0.84 | 7.27 | 0.42 ± 0.02 | 64.0 ± 9.6^{d} |

^{a}initial ratios
^{b}n = 3
^{c}volume-weighted Gaussian average
^{d}volume-weighted NICOMP average

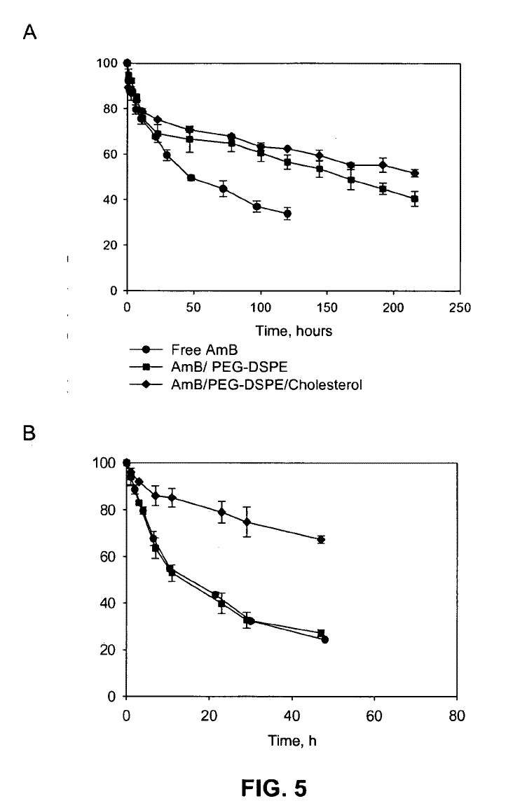

The absorbance spectrum for AmB (10 μg/mL) encapsulated in PEG-DSPE micelles in 10 mM HEPES buffer was sensitive to the initial AmB:PEG-DSPE ratio. At low levels of AmB incorporation (AmB:PEG-DSPE=1:2), three prominent bands were observed at 368, 388, and 417 nm (FIG. 4). At higher levels of AmB incorporation (AmB:PEG-DSPE=1:0.5), the band at 328 nm was prominent relative to higher wavelengths, indicative of AmB self-association. Incorporation of cholesterol into PEG-DSPE micelles (AmB:PEG-DSPE:Cholesterol=1:2:2) led to a narrowing of AmB absorption bands. The low peak I/IV ratio in the absorbance spectrum of AmB was characteristic of diminished self-aggregation.

FIG. 4 illustrates absorption spectra of AmB solubilized by PEG-DSPE and PEG-DSPE|Cholesterol micelles.

The width-at-half-height for peak IV in the AmB absorbance spectrum was estimated by fitting to a Gaussian curve of the form,

$$A = {A}_{0} + {A}_{max} \cdot exp ( \frac{- {( λ - {λ}_{max} )}^{2}}{2 {w}^{2}} )$$

where A₀ is the baseline absorbance value and Aₘₐₓ is the peak absorbance value, where λₘₐₓ is the wavelength corresponding to peak IV maximum, and where w is the width-at-half-height in nm.

Cholesterol incorporation shifted the λₘₐₓ from 417.2±0.04 nm in PEG-DSPE micelles to 415.1±0.03 nm in PEG-DSPE|Cholesterol micelles. Also, w decreased from 6.34±0.04 nm in PEG-DSPE micelles to 4.66±0.04 nm in PEG-DSPE|Cholesterol micelles. The origin of these spectral changes is not entirely clear as these may arise from either (1) selective affinity of AmB for cholesterol in PEG-DSPE micelles, or (2) restricted mobility of AmB in the micelle core due to structuring induced by cholesterol. Interaction between AmB and cholesterol in propanol-water mixtures has been suggested by Charbonneau et al. (25) and Barwicz et al. (26). However, Bolard et al. and Hsu Chen et al. have suggested that direct interaction between cholesterol and AmB in lipid vesicles is extremely weak, however that AmB has a greater affinity for structured lipids—an effect induced by cholesterol (3, 27). Fluorescence decay of a structurally related polyene, nystatin, has been correlated to changes in rigidity of DPPC vesicles containing varying levels of cholesterol (28). Regardless of its origin, addition of cholesterol to PEG-DSPE seems to influence the physical state of the encapsulated drug in the polymeric micelle.

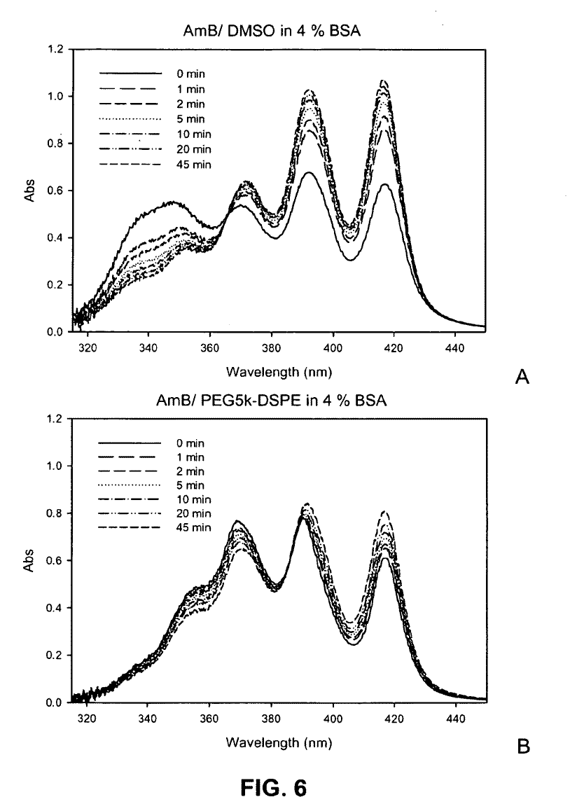

AmB release from PEG-DSPE micelles using equilibrium dialysis. AmB release from PEG-DSPE micelles was studied by using a dialysis setup as described in the literature (29, 30). FIG. 5 shows AmB release profiles (at 25° C. in a, 5 mM HEPES; and b, 4% BSA) and corresponding rate parameters are in Table 2. AmB release from PEG-DSPE and PEG-DSPE|Cholesterol micelles was slow (t_{1/2}=184, 259 h) in HEPES buffer, pointing to stable incorporation in the micelle core. In contrast, AmB is almost completely dissociated from D-AmB, which reflects a weak AmB—carrier interaction (4). Since AmB release from the dialysis cassette is limited by the equilibrium between monomeric and self-aggregated AmB, the release of AmB control was significantly slower in HEPES in which it is highly self-aggregated (CAC 1 μMA) compared with that in 4% BSA, which deaggregates AmB to some extent (CAC 10 μM). The t_{1/2} for release of free (or unencapsulated) AmB (30 μg/mL) using this setup was 69 h in HEPES and 18.6 h in 4% BSA.

AmB release from PEG-DSPE micelles in BSA was identical to that for free AmB (t_{1/2}=18.4 h). AmB release from PEG-DSPE|Cholesterol micelles in 4% BSA was significantly slower than AmB release from PEG-DSPE micelles (t_{1/2}=85.4 h). Our results are consistent with reports describing AmB incorporation in lipid vesicles containing cholesterol. Bolard et al. and HsuChen et al. have proposed that the incorporation of polyene antibiotics is favorable in ordered lipid bilayers such as those containing cholesterol (3, 27). Using absorption kinetics, Witzke et al. have estimated that the association of AmB in egg-PC vesicles with cholesterol was 35 times stronger than with similar vesicles without sterol (15). Our findings have determined that cholesterol in the context of polymeric micelles can yield slow AmB release. The slow release of deaggregated AmB from PEG-DSPE|Cholesterol micelles, in turn, can lower in vivo toxicity in comparison to either PEG-DSPE micelles without cholesterol or D-AmB.

TABLE 2
Fit parameters for AmB release from PEG-DSPE micelles at 25° C.

| | 5 mM HEPES | | | 4% BSA | | |
|---|---|---|---|---|---|---|
| | 100 · k | t_{1/2} | | 100 · k | t_{1/2} | |
| | (h⁻¹) | (h) | R² | (h⁻¹) | (h) | R² |
| Free AmB | 1.01 ± 0.10 | 68.5 | 0.94 | 3.72 ± 0.04 | 18.6 | 0.96 |
| AmB:PEG-DSPE 1:2 | 0.38 ± 0.04 | 183.9 | 0.90 | 3.75 ± 0.52 | 18.4 | 0.94 |
| AmB:PEG-DSPE:Cholesterol 1:2:2 | 0.27 ± 0.03 | 259.2 | 0.89 | 8.08 ± 0.08 | 85.8 | 0.94 |

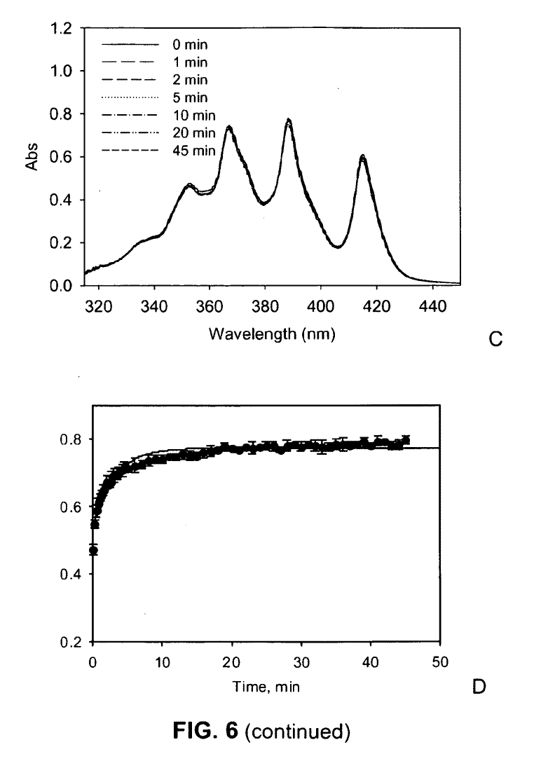

Electronic absorption kinetics in serum albumin. The electronic absorption spectrum of AmB is uniquely sensitive to the environment of the molecule. The absorbance at peak IV increases when AmB dissociates from either vesicles or micelles and associates with serum albumin. Thus, increases in the absorbance intensity at 412 nm have been used both to evaluate drug dissociation from PEG-DSPE micelles and to quantify apparent rate constants. FIGS. 6a-c show representative AmB absorbance spectra over time. FIGS. 6d-f show the corresponding changes in the absorbance at 412 nm. Kinetic rate parameters that were obtained by fitting the absorbance values in FIGS. 6d-f to a first-order process are shown in Table 3.

TABLE 3
Fit parameters for absorbance change for PEG-DSPE incorporated
AmB at 412 nm upon dilution in 4% BSA (FIGS. 6d-f)

| | 100 k (min⁻¹) | t_{1/2-1} (min) | R² | A_{∞} |
|---|---|---|---|---|
| Unencapsulated AmB | 3.25 ± 0.23 | 2.1 | 0.94 | 0.77 |
| AmB:PEG-DSPE 1:2 | 6.63 ± 0.53 | 10.46 | 0.98 | 0.56 |
| AmB:PEG-DSPE:Cholesterol | — | — | — | 0.42 |
| 1:2:2 | | | | |

The addition of free AmB to 4% BSA resulted in an initial broad band at ca. 350 nm, characteristic of self-aggregated AmB (FIG. 6a). The intensity of this band diminished over time, with increasing intensity at higher wavelengths. This finding is consistent with drug deaggregation in 4% BSA (31). The half-life for this association was estimated as 2.1 min, assuming first-order kinetics. Peak IV absorbance of AmB encapsulated in PEG-DSPE micelles increased over 45 min upon dilution in 4% BSA and absorbance maxima for peaks III and IV underwent minimal shifts (˜2 nm) (FIG. 6b). The dissociation half-life, assuming first-order kinetics was estimated as 10.5 min. This result was consistent with rapid AmB release from PEG-DSPE micelles in the presence of 4% BSA observed in dialysis studies. In contrast, there were minimal changes in the absorbance spectrum of AmB in PEG-DSPE|Cholesterol micelles in serum albumin (FIG. 6c). This finding indicates that AmB that was incorporated in PEG-DSPE|Cholesterol micelles was not free to interact with serum albumin. The slow release of AmB from these micelles occurs presumably through a simple diffusion process.

FIG. 6 illustrates absorbance spectra of micelle encapsulated AmB in 4% BSA. a. Unencapsulated AmB (in DMSO) b. AmB in PEG-DSPE micelles (AmB: PEG-DSPE=1:2) c. AmB in PEG-DSPE|Cholesterol micelles (AmB: PEG-DSPE: Cholesterol=1:2:2) d-f. Absorbance at 412 nm for as a function of incubation time corresponding to panels a-c. AmB concentration was 10 μg/mL.

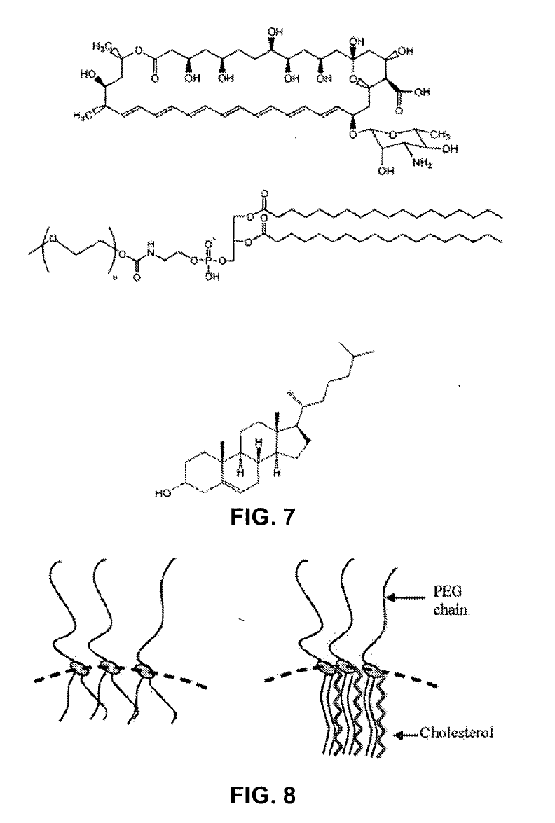

Conclusions. PEG-DSPE|Cholesterol micelles prepared using a solvent evaporation method could solubilize high levels of deaggregated AmB. The co-incorporation of cholesterol increases the rigidity of cores of PEG-DSPE micelles, possibly due to a structuring effect induced by hydrophobic interaction (see FIG. 8 for a diagram of such structuring effect). The narrowed bands in the AmB absorption spectrum indicated that cholesterol alters the association of AmB with the PEG-DSPE micelle core. Absorption kinetics and dialysis experiments indicate that while AmB in PEG-DSPE is free to interact with serum albumin, the drug is more stably incorporated in PEG-DSPE|Cholesterol micelles. Therefore the potentially greater interaction of the polyene antibiotic with structured lipids has been utilized to develop compositions and methods for controlled release.

### References for Example 1

1. Vakil, R., Kwon, G., PEG-phospholipid micelles for the delivery of amphotericin B. J. Controlled Release, 2005. 101(1-3): p. 386-9.
2. Barwicz, J., Tancrede, P., The effect of aggregation state of amphotericin B on its interactions with cholesterol- or ergosterol-containing phosphatidylcholine monolayers. Chem. Phys. Lipids, 1997. 85(2): p. 145-55.
3. Bolard, J., Legrand, P., Heitz, F., Cybulska, B., One-sided action of amphotericin B on cholesterol-containing membranes is determined by its self-association in the medium. Biochemistry, 1991. 30(23): p. 5707-15.
4. Brajtburg, J., Bolard, J., Carrier effects on biological activity of amphotericin B. Clin. Microbiol. Rev., 1996. 9(4): p. 512-31.
5. Gruda, I., Dussault, N., Effect of the aggregation state of amphotericin B on its interaction with ergosterol. Biochem. Cell Biol., 1988. 66(3): p. 177-83.
6. Imhof, A., Walter, R. B., Schaffner, A., Continuous infusion of escalated doses of amphotericin b deoxycholate: an open-label observational study. Clin. Infect. Dis., 2003. 36: p. 943-951.
7. Eriksson, U., Seifer, B., Schaffner, A., Comparison of effects of amphotericin b deoxycholate infused over 4 to 24 hours: Randomized controlled trial. Br. Med. J., 2001. 322: p. 579-582.
8. Peleg, A., Woods, M., Continuous and 4 h infusion of amphotericin B: a comparative study involving high-risk haematology patients. J. Antimicrob. Chemother., 2004. 54(4): p. 803-8.
9. Hiemenz, J., Amphotericin B deoxycholate administered by continuous infusion: does the dosage make a difference? Clin. Infect. Dis., 2003. 36(8): p. 952-3.
10. Schulenburg, A., Sperr, W., Rabitsch, W., Knobl, P., Thalhammer, F., Brief report: Practicability and safety of amphotericin b deoxycholate as continuous infusion in neutropenic patients with hematological malignancies. Leukemia & Lymphoma, 2005. 46: p. 1163-1167.
11. Ohvo-Rekila, H., Ramstedt, B., Leppimaki, P., Slotte, J., Cholesterol interactions with phospholipids in membranes. Prog. Lipid Res., 2002. 41(1): p. 66-97.
12. Lavasanifar, A., Samuel, J., Kwon, G., The effect of alkyl core structure on micellar properties of poly(ethylene oxide)-block-poly(L-aspartamide) derivatives. Colloids Surf. B, 2001. 22(2): p. 115-126.
13. Winnik, F., Photophysics of preassociated pyrenes in aqueous polymer solutions and in other organized media. Chem. Rev., 1993. 93(2): p. 587-614.
14. Claridge, T., High-Resolution NMR Techniques in Organic Chemistry. 1999: Pergamon.
15. Witzke, N., Bittman, R., Dissociation kinetics and equilibrium binding properties of polyene antibiotic complexes with phosphatidylcholine/sterol vesicles. Biochemistry, 1984. 23(8): p. 1668-74.
16. Beggs, W., Antioxidant-stabilized amphotericin B. Diagn. Microbiol. Infect. Dis., 1983. 1(4): p. 339-341.
17. Wolszczak, M., Miller, J., Characterization of non-ionic surfactant aggregates by fluorometric techniques. J. Photochem. Photobiol. A, 2002. 147(1): p. 45-54.
18. Mcconnell, H., Radhakrishnan, A., Condensed complexes of cholesterol and phospholipids. Biochim. Biophys. Acta, 2003. 1610(2): p. 159-73.
19. Davies, M., Schuster, H., Brauner, J., Mendelsohn, R., Effects of cholesterol on conformational disorder in dipalmitoylphosphatidylcholine bilayers. A quantitative IR study of the depth dependence. Biochemistry, 1990. 29(18): p. 4368-73.
20. Hofsass, C., Lindahl, E., Edholm, O., Molecular dynamics simulations of phospholipid bilayers with cholesterol. Biophys. J., 2003. 84(4): p. 2192-206.
21. Kwon, G., Kataoka, K., Block copolymer micelles as long-circulating drug vehicles. Adv. Drug Del. Rev., 1995. 16(2-3): p. 295-309.
22. Montesano, G., Bartucci, R., Belsito, S., Marsh, D., Sportelli, L., Lipid membrane expansion and micelle formation by polymer-grafted lipids: scaling with polymer length studied by spin-label electron spin resonance. Biophys. J., 2001. 80(3): p. 1372-83.
23. Hristova, K., Needham, D., Phase behavior of a lipid/polymer-lipid mixture in aqueous medium. Macromolecules, 1995. 28: p. 991-1002.
24. Belsito, S., Bartucci, R., Montesano, G., Marsh, D., Sportelli, L., Molecular and mesoscopic properties of hydrophilic polymer-grafted phospholipids mixed with phosphatidylcholine in aqueous dispersion: interaction of dipalmitoyl N-poly(ethylene glycol)phosphatidylethanolamine with dipalmitoylphosphatidylcholine studied by spectrophotometry and spin-label electron spin resonance. Biophys. J., 2000. 78(3): p. 1420-30.
25. Charbonneau, C., Fournier, I., Dufresne, S., Barwicz, J., Tancrede, P., The interactions of amphotericin B with various sterols in relation to its possible use in anticancer therapy. Biophys. Chem., 2001. 91(2): p. 125-33.
26. Barwicz, J., Gareau, R., Audet, A., Morisset, A., Villiard, J., Gruda, I., Inhibition of the interaction between lipoproteins and amphotericin B by some delivery systems. Biochem. Biophys. Res. Comm., 1991. 181(2): p. 722-728.
27. Hsu Chen, C., Feingold, D., Polyene antibiotic action on lecithin liposomes: effect of cholesterol and fatty acyl chains. Biochem. Biophys. Res. Commun., 1973. 51(4): p. 972-8.
28. Castanho, M., Coutinho, A., Prieto, M., Absorption and fluorescence spectra of polyene antibiotics in the presence of cholesterol. J. Biol. Chem., 1992. 267(1): p. 204-9.
29. Forrest, M., Won, C., Malick, A., Kwon, G., In vitro release of the mTOR inhibitor rapamycin from poly(ethylene glycol)-b-poly(epsilon-caprolactone) micelles. J. Controlled Release, 2006. 110(2): p. 370-7.
30. Lim Soo, P., Luo, L., Maysinger, D., Eisenberg, A., Incorporation and release of hydrophobic probes in biocompatible polycaprolactone-block-poly(ethylene oxide) micelles: Implications for drug delivery. Langmuir, 2002. 18(25): p. 9996-10004.
31. Aramwit, P., Yu, B., Lavasanifar, A., Samuel, J., Kwon, G., The effect of serum albumin on the aggregation state and toxicity of amphotericin B. J. Pharm. Sci., 2000. 89(12): p. 1589-1593.

### Example 2

### Polymeric Micelles for Combination Antifungal Therapy

In an embodiment, the invention provides compositions and methods relating to combinations of pharmaceutical agents and combination therapies. In an embodiment, the combinations relate to at least one antifungal agent. In an embodiment, the combinations relate to at least two antifungal agents. In an embodiment, the combinations relate to at least three antifungal agents. In an embodiment, at least one antifungal agent is Amphotericin B. In an embodiment, a combination involves the fungal agent rapamycin and/or 5-FC. In an embodiment, a combination involves Diflucan® (fluconazole).

Abstract. Combination antifungal therapy involving amphotericin B (AmB) has been restricted by poor physical compatibility with drugs and vehicles, especially when solubilized as a colloidal dispersion with sodium deoxycholate (D-AmB). We have formulated AmB in PEG-DSPE micelles using a solvent evaporation method. Significantly, AmB in this form is stable against precipitation in a saline solution over prolonged periods, enabling slow infusion and simultaneous sodium supplementation, which have been shown to reduce AmB related toxicity. Rapamycin and 5-fluorocytosine are antifungal agents with unique mechanisms of activity, with potential for cooperative interaction with AmB. AmB and rapamycin encapsulated in PEG-DSPE micelles retain potent in-vitro activity, against test fungal isolates. In an embodiment, this novel formulation involving PEG-DSPE micelles enables combination of the three potent antifungals in a saline vehicle. We have investigated interactive effects of pair-wise and three-drug combinations in-vitro using a checkerboard method. Our results indicate a cooperative increase in antifungal potency when PEG-DSPE micelle encapsulated AmB is combined with rapamycin and 5-FC. Since the three-drug combination is stable against precipitation, this represents a significant advance for efficacy of the three-drug combination, optionally in conjunction with sodium supplementation and/or slow infusion via a single intravenous access-line.

### 1. Introduction

The increasing incidence of hospital acquired opportunistic fungal infections is of concern, particularly in the context of a growing population of patients with compromised or suppressed immune systems (1). Amphotericin B solubilized as a colloidal dispersion by sodium deoxycholate (D-AmB) is a first-line antifungal agent administered to most patients with invasive candidiasis, despite severe kidney toxicity (2). Liposomal amphotericin B (L-AmB) is less toxic compared to D-AmB allowing for dose-escalation and improved tolerability; however, L-AmB has lower antifungal activity at equal doses necessitating significantly higher doses for comparable efficacy, raising doubts about a meaningful increase in the therapeutic index (3). Notably, L-AmB has not had a significant impact on the crude or attributable mortality compared to D-AmB (4).

There is a renewed interest in studying combinations of antifungal drugs to explore one or more potential advantages such as broad-spectrum efficacy, indifferent or synergistic potency, improved safety and tolerability and activity targeted against resistant organisms (5, 6). There is growing evidence that the combination of D-AmB and 5-fluorocytosine (5-FC) has better activity against candidiasis compared to D-AmB (7-10). The in vitro antifungal effects of the combination of D-AmB and 5-FC are synergistic or indifferent, depending on the species of Candida (6). It is hypothesized that the mechanism by which AmB potentiates 5-FC activity is to increase the penetration of 5-FC across fungal membranes, highlighting the value of exposing the fungal pathogen to 5-FC at the same time or after exposure, to AmB (11). Recently, D-AmB in 5% dextrose and 5-FC in 0.9% NaCl, injected intraperitoneally as separate and rapid sequential injections showed additive activity in a murine model of candidiasis according to the response-surface model (12).

Rapamycin exerts potent antifungal activity by inhibiting TOR kinases (MIC against C. albicans <0.02 mg/L) (13, 14). Early animal experiments showed effectiveness against Candida infection, however, enthusiasm for use as an antifungal agent were lowered on emergence of potent immunosuppression in hosts (15). Rapamycin-analogues that selectively bind yeast TOR kinases present a thousand-fold reduction in immunosuppressive activity, while retaining some measure of antifungal activity (MIC <3 mg/L for C. albicans and C. glabrata) (16). The TOR signaling cascade represents a conserved pathway across yeast, involved in eliciting cell response to a wide variety of stimuli including nutrients and external stress (17). Considering the unique mechanism of action, this class of antifungal antibiotics has unexplored potential for treatment of systemic fungal disease. Simultaneous exposure of pathogenic fungi to AmB induced cationic stress and inhibition of yeast survival pathways by rapamycin may represent opportunity for cooperative increase in antifungal potency. However, rapamycin is very poorly water-soluble and has proven to be highly challenging for drug solubilization requiring analogues for clinical trials in cancer.

Several strategies have emerged which are beneficial in lowering AmB related toxicities. There is recent clinical evidence which suggests that D-AmB administered as a continuous infusion over 24 h results in a lower incidence of nephrotoxicity and infusion-related side-effects compared to the standard 2-4 h infusion at an equal dose (18-20). Although the mechanism for lowered toxicities is poorly understood, it is proposed that slow infusion results in lower levels of protein-bound drug capable of evoking host toxicity, compared to the standard infusion regimen (21). Saline loading during AmB therapy has been shown to reduce the severity of AmB related toxicities. In a double-blind, placebo-controlled study with human subjects, sodium supplementation has been shown to diminish infusion-related side effects and nephrotoxicity caused by D-AmB (22).

Combination antifungal therapy involving AmB has been restricted by factors such as poor physical stability and compatibility with antifungal drugs and vehicles, especially in the form of D-AmB. D-AmB is not compatible with saline and precipitates instantly on dilution (23). Although saline loading has gained some degree of acceptance, this procedure necessitates sequential administration of saline and D-AmB, and special care must be taken to adequately flush infusion lines with 5% dextrose prior to D-AmB administration to avoid potentially hazardous drug precipitation. Multiple-agent therapy with continuous administration of D-AmB would require additional intravenous (IV) access-lines owing to incompatibility, raising concern of increased risk of infection in these critically ill patients.

We have previously reported that micelles formed from the amphiphilic polymer 1,2-Distearoyl-sn-glycero-3-phosphoethanolamine-N-methoxy poly(ethylene glycol) (PEG-DSPE) readily solubilize AmB (AmB|PEG-DSPE) (24). In this work, we report that PEG-DSPE micelles also encapsulate high levels of rapamycin when prepared using an identical solvent evaporation method (rapamycin|PEG-DSPE). AmB is stably incorporated in PEG-DSPE micelles in a form which is compatible with rapamycin and 5-FC in 0.9% NaCl. We have evaluated the in vitro activity of two- and three-drug combinations using a broth microdilution method. Interactive effects have been quantified by two methods used in the literature: FIC index, combination index calculated using median dose-effect equation.

### 2. Materials

AmB was obtained as a gift from Alpharma (Copenhagen, Denmark). Rapamycin and 5-FC were purchased from LC Labs (Wobum, Mass.) and Sigma (St. Louis, Mo.), respectively. AmB formulated in sodium deoxycholate (conventional AmB, D-AmB) was purchased from Sigma (St. Louis, Mo.). The drugs were stored at −20° C. until use. PEG-DSPE (Mₙ=5800 g/mol) was obtained from Avanti Polar Lipids (Alabaster, Ala.). All other reagents used were of analytical grade and were used without further purification.

### 3. Micelle Preparation and Incorporation of AmB and Rapamycin

PEG-DSPE (6.0 mg/mL in chloroform) was mixed with AmB (0.25 mg/mL in methanol) or rapamycin (1 mg/mL in chloroform) in a round bottom flask. The organic solvent was evaporated under vacuum to produce a thin film of co-precipitated drug and polymer. This film was dissolved in 10 mM HEPES, pH 7.0 and incubated at room temperature for 10 min to allow for complete equilibration. The micellar solution was filtered through a 0.45-μm polyethersulfone (PES) filter. Empty micelles were prepared using an identical procedure without drug. The concentration of AmB was quantified by diluting a 50 μL aliquot of AmB in 1.95 mL DMF and observing absorbance at 413.5 nm. This assay was tested for linearity in the 0.02-0.8 mg/mL range. For determination of rapamycin content, 5 μL samples were injected into 4.6 mm×50 mm Ace 3 C18 reversed-phase column and absorbance detected at 277 nm. The column was maintained at 25° C. and eluent flow rate was set at 1 mL/min. The eluent composition was linearly varied by mixing methanol and a 1:1 methanol−50 mM acetic acid mixture over 4 min. The assay was tested for linearity in the 0.1-100 μg/mL range.

### 4. Micelle Characterization

### 4.1 Dynamic Light Scattering

Particle sizes were determined using dynamic light scattering using the NICOMP ZLS380 particle sizer (Particle Sizing Systems, Santa Barbara, Calif.) equipped with a 639 nm laser at a fixed angle of 90°. Data was acquired to have greater than 100 k counts in Channel 1. The light scattering data was interpreted using Gaussian and NICOMP analyses. The NICOMP analysis permits deconvolution of light scattering data into multimodal distributions. Particle sizes were expressed as volume-weighted diameters.

### 4.2 Size Exclusion Chromatography

Aqueous micellar solutions were diluted to 1.0 mg/mL polymer content in water. 100 μL samples were injected in triplicate onto a Shodex PROTEIN KW-804 size exclusion column (Showa Denko, Japan). The column was equilibrated using water. The flow rate was maintained at 0.75 mL/min and the column compartment was maintained at 10° C. The elution of empty polymeric micelles was monitored using refractive index. Elution of polymeric micelle incorporated AmB was detected using absorbance at 412 nm.

### 5. Solution Turbidity Measurements

Precipitation of D-AmB was studied by observing solution turbidity (OD₆₅ₙₘ) using a Cary 50 UV spectrophotometer equipped with a dip probe.

Aqueous solutions containing 0.9% NaCl or 5% dextrose were maintained at 25° C. in a water-jacketed beaker. OD₆₅₀ₙₘ was observed for the initial 2.0 min to estimate baseline turbidity. Thereafter, D-AmB was added to aqueous medium and changes in OD₆₅₀ₙₘ were observed over the duration of the experiment. The final concentration of AmB was 0.1 mg/mL. Changes in solution turbidity on the addition of AmB|PEG-DSPE to 0.9% NaCl and 0.9% dextrose were similarly measured. Further, the potential for precipitation on mixing AmB|PEG-DSPE, rapamycin|PEG-DSPE and 5-FC was examined using an identical procedure.

### 6. In Vitro Susceptibility of Fungal Isolates

### 6.1 Estimation of Minimum Inhibitory Concentration (MIC)

C. albicans 98-17 and C. albicans 98-234 were maintained on SDA plates. Susceptibility of yeast isolates was performed using broth microdilution in RPMI-1640 (supplemented with 0.165 M morpholinepropanesulfonic acid, buffered to pH 7.0) according to procedures recommended in NCCLS M27-A2 (25). Test drug solutions were incubated with yeast inoculum at 35° C. in 96-well plates for a 24 h period. All measurements were done in triplicate. Percent yeast growth relative to drug-free control was determined by measuring optical density (OD) at 650 nm, using the Microplate EL 312e plate reader. Minimum inhibitory concentration (MIC) values represent drug concentrations which resulted in >95% inhibition of fungal growth relative to drug-free control. The MIC values were expressed as a mean of three determinations.

### 6.2 Interactive Effects of Drug-Combinations

The interactive effects of drug combinations were assessed using a checkerboard layout. For a two-drug combination the concentration of each drug was varied, for a constant level of the other drug. For a three-drug combination, the levels of 5-FC were varied for a constant ratio of AmB|PEG-DSPE:rapamycin|PEG-DSPE (1:2 or 2:1). Determination of fungal growth in response to drug combinations was performed in triplicate. Interactive effects were interpreted using two methods extensively used in the literature: FIC analysis (6) and calculation of a combination index using a median-effect equation (26, 27).

FIC analysis:

The FIC index (FICI) for a three drug combination is defined by the equation:

$$FICI = \frac{{MIC}_{A - combination}}{{MIC}_{A}} + \frac{{MIC}_{B - combination}}{{MIC}_{B}} + \frac{{MIC}_{C - combination}}{{MIC}_{C}}$$

where, MIC_{A-combination} and MIC_{A} represent the concentration of “drug A” which leads to greater than 95% inhibition of fungal growth in combination with “drug B” and “drug C”, and as a single agent, respectively. The FICI values have been interpreted to indicate synergism, additivity or antagonism according to the recommendations in the literature (5, 28-30) for the interpretation of FIC index values and have been shown in Table 4.

### Combination Indices Using Median-Effect Response:

Calculation of a combination index was done in two steps. First, inhibition of fungal growth in response to varying concentrations of single-drug was fit to median-effect function:

$${f}_{a} = \frac{1}{1 + {( {D}_{m} / D )}^{m}}$$

where fₐ is the mean growth inhibition, relative to drug-free control, Dₘ is the drug concentration for a 50% growth inhibition, and m is the slope-factor describing the dose-response curve. Parameter Dₘ and m for each drug were estimated by non-linear regression using SigmaPlot (v. 9.0).

The combination index for was estimated on the assumption of mutually exclusive effects using the CombiTool software using the following equation:

$$CI = \frac{{D}_{A - combination}}{{D}_{A , fa}} + \frac{{D}_{B - combination}}{{D}_{B , fa}} + \frac{{D}_{C - combination}}{{D}_{C , fa}}$$

where D_{A-combination}, D_{B-combination} and D_{C-combination} are concentrations of “drug A”, “drug B” and “drug C” which cause growth inhibition, fₐ in combination. D_{A,fa}, D_{B,fa} and D_{C,fa} are corresponding drug concentrations which produce fₐ inhibition alone and were calculated using the median-effect equation, knowing Dₘ and m. Combination indices were represented as color maps using Origin software (version 7.0).

### 7. Results and Discussion

### 7.1 Micelle Characterization

AmB could be efficiently solubilized by PEG-DSPE micelles (AmB|PEG-DSPE) using the solvent evaporation method, leading to solubilization of this poorly water soluble drug (Table 1). Additionally, incorporation of AmB in PEG-DSPE micelles resulted in drug deaggregation owing to polymer—drug interactions (24). High levels of rapamycin could be loaded into PEG-DSPE micelles (rapamycin|PEG-DSPE) using a similar procedure. The level of rapamycin solubilized was 0.38 mg/mL, with a high yield (97% of initial drug). Using dynamic light scattering, the size of AmB|PEG-DSPE micelles (PEG-DSPE: AmB=2.0) was determined to be 19.3±4.4 nm, slightly larger than empty PEG-DSPE micelles (16.1±2.2 nm). The size of rapamycin|PEG-DSPE micelles was similarly small, 26.4±2.0 nm.

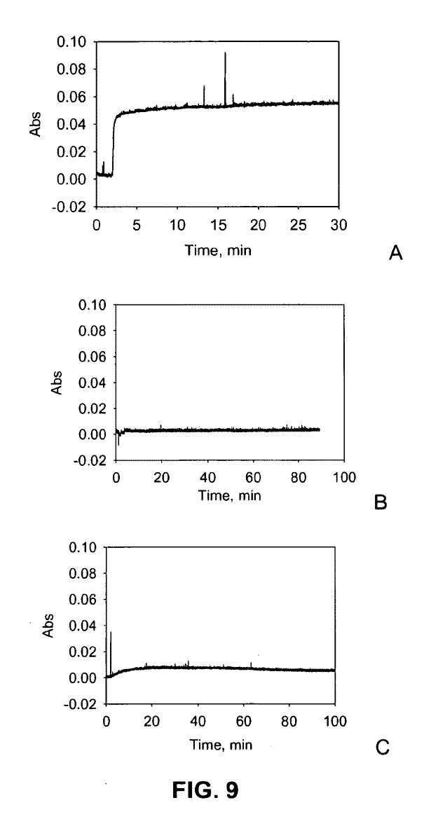

FIG. 9. Light scattering at 650 nm on admixing a. conventional AmB (D-AmB) with 0.9% NaCl b. D-AmB with 5% dextrose c. AmB|PEG-DSPE with rapamycin/PEG-DSPE and 5-FC in 0.9% NaCl. The final AmB concentration was 0.1 mg/mL

TABLE 1
Solubilization of AmB and Rapamycin in PEG-DSPE micelles

| | | | Level of | |
|---|---|---|---|---|
| | Fraction of | Drug | drug | |
| | initial drug | loading | solubilized, | Diameter |
| | encapsulated | % w/w | mg/mL | (nm)^{a} |
| PEG-DSPE | — | — | — | 16.1 ± 2.2 |
| AmB\|PEG-DSPE | 0.78 | 7.21 | 0.38 ± 0.12 | 19.3 ± 4.4 |
| Rapamycin\|PEG- | 0.97 | 7.76 | 0.37 ± 0.06 | 26.4 ± 2.0 |
| DSPE | | | | |

^{a}volume-weighted Gaussian average

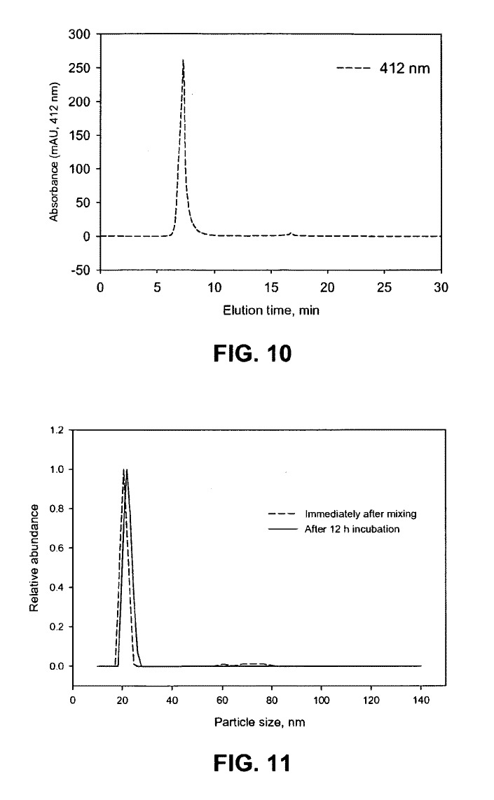

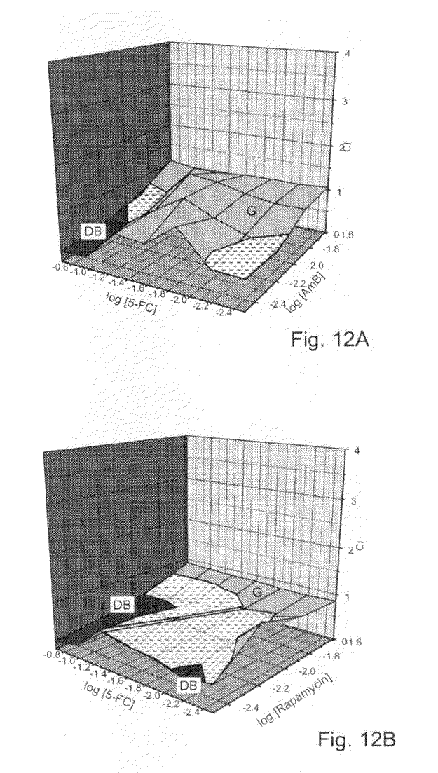

We have studied changes in solution turbidity on addition of D-AmB to 0.9% NaCl or 5% dextrose solutions using a spectrophotometer equipped with a dip-probe assembly. There was an instantaneous increase in solution turbidity on addition of D-AmB to 0.9% NaCl, indicative of rapid precipitation on mixing (FIG. 9-a). Addition of D-AmB to 5% dextrose did not result in increased solution turbidity, consistent with the conventional use of 5% dextrose as acceptable vehicle for dilution of D-AmB (FIG. 9-b). In contrast, addition of AmB|PEG-DSPE and rapamycin|PEG-DSPE to 5-FC in 0.9% NaCl led to a minor increase in OD₆₅₀ nm, attributed to light scattering by intact PEG-DSPE micelles (FIG. 9-c). AmB eluted with intact PEG-DSPE micelles with no appearance of free AmB, when incubated with rapamycin|PEG-DSPE and 5-FC in saline at 37° C. for several hours, indicating that AmB is stably solubilized in PEG-DSPE micelles (FIG. 10). Results from dynamic light scattering indicated negligible changes in the size of AmB|PEG-DSPE micelles several hours after mixing with rapamycin|PEG-DSPE and 5-FC in 0.9 NaCl. Importantly, this study did not find drug aggregates in solution (FIG. 11).

FIG. 10. SEC of AmB|PEG-DSPE, rapamycin|PEG-DSPE and 5-FC in 0.9% NaCl^{a,b} (where: ^{a}incubation at 37° C. for 4.5 h. ^{b} 40 ᵤ g/mL AmB|PEG-DSPE, 40 μg/mL rapamycin|PEG-DSPE, 2 mg/mL 5-FC). Column: Shodex PROTEIN-KW804. Eluent: Water at 0.75 mL/min. Column temperature: 10° C.

FIG. 11. Sizing of AmB|PEG-DSPE, rapamycin|PEG-DSPE and 5-FC mixture in 0.9% NaCl using DLS. a. immediately after mixing b. after incubation for 12 h at room temperature.

These results are particularly relevant from a delivery prespective, since AmB|PEG-DSPE can be suitable for slow infusion over several hours without concern for drug precipitation on dilution. In this form, AmB has a distinct advantage over D-AmB, since the polymeric micelle form of AmB may allow for simultaneous administration of saline via a single IV access-line. Since AmB|PEG-DSPE remains solubilized when mixed with 5-FC and solubilized rapamycin, there can be a benefit of drug combinations over monotherapy.

### 7.2 In Vitro Susceptibility Studies

In vitro susceptibility of C. albicans 98-17 and C. albicans 98-234 was evaluated using the broth microdilution method. MIC values for AmB|PEG-DSPE were 0.05 μg/mL. The MIC for D-AmB, in which the drug is highly self-aggregated, was 0.25 μg/mL (Table 2).

TABLE 2
Minimum Inhibitory Concentrations (MIC)
in mg/L against C. albicans isolates

Strain of C. albicans

| | 98-17 | 98-234 |
|---|---|---|
| D-AmB | 0.25 | 0.25 |
| AmB\| PEG-DSPE | 0.05 | 0.05 |
| Rapamycin\| PEG-DSPE | 0.05 | 0.05 |
| 5-FC | 0.1 | 0.1 |

PEG-DSPE did not exhibit intrinsic antifungal activity (MIC >10 μg/mL). The reason for enhanced activity of PEG-DSPE encapsulated AmB is unclear—however a similar potentiation of AmB activity has been demonstrated on encapsulation in mixed micelles formed from poly(ε-caprolactone) and poloxamer 188 (31). We hypothesize that in contrast with D-AmB that produces a mixture of monomers and water soluble aggregates, AmB dissociated from intact PEG-DSPE micelles is predominantly in a monomeric form, which results in a higher number of membrane-active units compared to the D-AmB. The MIC of rapamycin in PEG-DSPE micelles was 0.05 mg/L, comparable to that for free rapamycin reported in the literature (14). The MIC for 5-FC, corresponding to complete inhibition of fungal-growth was 0.1 mg/L—consistent with some literature reports (10, 32).

TABLE 3
$Best fit parameters to median \text{-} effect equation , {f}_{a} = \frac{1}{1 + {( {D}_{m} / D )}^{m}}$

| | Strain of Candida albicans | | | |
|---|---|---|---|---|
| | 98-17 | | 98-234 | |
| | Dₘ × 10³ | m | Dₘ × 10³ | m |
| AmB\|PEG-DSPE | 17.3 ± 0.2 | 6.0 ± 0.2 | 11.3 ± 0.4 | 6.5 ± 0.5 |
| Rapamycin\|PEG-DSPE | 32.3 ± 2.2 | 6.5 ± 2.2 | 24.2 ± 0.9 | 3.5 ± 0.5 |
| 5-FC | 70.7 ± 7.3 | 2.0 ± 0.8 | 39.8 ± 0.1 | 1.9 ± 0.4 |

Table 3 shows parameters obtained by fitting the dose-response curve for AmB|PEG-DSPE, rapamycin|PEG-DSPE or 5-FC to the median-effect equation. Reasonable fits to the data were obtained using non-linear regression, judged by R² values greater than 0.9. Dₘ represents drug potency, corresponding to the drug concentration for 50% inhibition and the slope-factor m represents the shape of the dose-response curve. Dₘ values for AmB|PEG-DSPE and rapamycin|PEG-DSPE were low which were consistent with low MIC values. A high value for the shape factor, m for both AmB|PEG-DSPE and rapamycin|PEG-DSPE implied transition from no inhibition to complete growth inhibition over a narrow range of concentration. Dₘ for 5-FC varied from 0.04 to 0.07 mg/L, depending on the isolate tested and the shape factor ranged from 1.9 to 2.0, lower in comparison with AmB|PEG-DSPE and rapamycin|PEG-DSPE.

We have studied interactive effects of AmB|PEG-DSPE, rapamycin|PEG-DSPE and 5-FC as two- and three-drug combinations using a checkerboard layout. The complexity of assessing drug interactions has been stressed in the literature (5, 10, 33). Calculation of FIC indices have been most extensively used for evaluating interactive effects between antimicrobial combinations. A limitation of the FIC analysis is that this analysis presumes that drug interactions are unvariant and apply across all concentrations. Several methods have been proposed to study drug interactions over an appropriate concentration range and have been reviewed in detail by Greco (34). The response surface method proposed by Greco et al. (35) and the method of Chou and Talalay using the median-effect equation (26, 30) have been frequently used in studying antiviral and antineoplastic drug interactions, each with a set of important underlying assumptions and limitations. An advantage of the method of Chou and Talalay is that it allows visualization of the combination index for different drug ratios—an important goal in studying drug combinations, in vitro. This approach has particular utility when the drug combination under study shows cooperativity for some ratios and antagonism at other ratios. We have calculated combination indices for two- and three-drug combinations, based on the median-effect equation using parameters in Table 3.

Consistent variations of FICI or combination index from 1 are taken as measures of drug interaction. Although a subject of debate, there is general consensus that a synergistic interaction between drugs is claimed for FICI <0.5. Similarly, drug antagonism is claimed for FICI values greater than 4 (5, 28, 29). Additionally, we have used the terms “moderate synergy” or “moderate antagonism” in the following discussion according to recommendations of Chou (30). The graphs have been color-coded to represent synergism (dark blue, indicated in figures with “DB”), moderate synergism (light blue, indicated with “LB” and dashed pattern), indifference (green, indicated with “G”) and antagonism (red, indicated with “R”), according to the ranges in Table 4.

TABLE 4
Range of combination index (CI) or fractional inhibitory
concentration (FIC) index and associated descriptions

| Range of CI or FIC | Description |
|---|---|
| <0.5 | Synergism |
| 0.5-0.75 | Moderate synergism |
| 0.75-1.5 | Indifference/Additivity |
| 1.5-4 | Moderate Antagonism |
| >4 | Antagonism |

The FICI for the AmB|PEG-DSPE-rapamycin|PEG-DSPE combination ranged from 1.1-1.6 (Table 5), suggesting an indifferent interaction between the antifungal agents. It is noted that a similar indifference has been reported for D-AmB and unformulated rapamycin against Aspergillus fumigatus isolates (36). The lack of antagonism between drugs is an important result since drug combinations would allow for an increase in antifungal activity in situations when dose-escalation is not an option. Mechanistic studies of drug-interactions were outside the scope of this work—however, it appears that these antifungal drugs act by relatively independent mechanisms. This combination can present a strategy which combines the rapid fungicidal action of AmB|PEG-DSPE with low doses of rapamycin|PEG-DSPE, which may work well as consolidation or clearance therapy.

TABLE 5
Fractional inhibitory concentration (FIC) analysis for test isolates
against AmB - Rapamycin - 5-FC combinations after 24 h.

| | C. albicans | C. albicans |
|---|---|---|
| | 98-17 | 98-234 |
| AmB\|PEG-DSPE - | 1.3 (1.1-1.6) | 1 |
| Rapamycin\|PEG-DSPE | | |
| AmB\|PEG-DSPE 5-FC | 0.6 | 0.8 |
| Rapamycin\|PEG-DSPE - 5-FC | 1 | 1 |
| AmB\|PEG-DSPE | 0.7 (0.4-0.8) | 0.45 (0.4-0.5) |
| Rapamycin\|PEG-DSPE - 5-FC | | |

Results are expressed as mean (range) for test-replicates.

The 5-FC-AmB combination has been extensively studied in vitro and in animal models (5, 12). The FICI values for the 5-FC-AmB|PEG-DSPE ranged from 0.6-0.8, for C. albicans 98-17 and 98-234, indicating that this combination exerted moderately synergistic behavior. The varying nature of the interaction is consistent with earlier reports that indicate that the D-AmB-5-FC interaction is variable and depends on experimental conditions and on the isolate tested (10, 33). A majority of the isolates studied in the literature reported indifferent activities, whereas the interaction was synergistic for some isolates. Moderate antagonism has been reported in a small fraction of C. albicans isolates tested (37).

Drug combinations with high proportion of 5-FC relative to AmB|PEG-DSPE had combination index values less than 0.5 indicating synergistic potential. The mechanism of cooperative interaction between 5-FC and AmB|PEG-DSPE is not well understood, however, higher penetration of 5-FC through the yeast cell-membrane in the presence of D-AmB has been proposed. It is noted that nystatin, structurally related to AmB exerted an antagonistic interaction with 5-FC (38). Alternatively, D-AmB is thought to influence processes that transport 5-FC out of the yeast cells (5).

The combination of 5-FC with rapamycin|PEG-DSPE was indifferent with FICI equal to 1 for the three isolates tested. The combination index analysis indicated a trend similar to the 5-FC-AmB|PEG-DSPE combination, with a moderately synergistic interaction at high 5-FC: rapamycin|PEG-DSPE ratios for C. albicans 98-17 and 98-234. This aspect of the drug interaction was missed by the FIC analysis, which concludes additivity based on concentrations required to completely inhibit fungal growth.

The three-drug combination of 5-FC, AmB|PEG-DSPE and rapamycin|PEG-DSPE resulted in a moderately synergistic interaction for C. albicans 98-17 and 98-234, with the FICI ranging from 0.4 to 0.8, consistently lower than 1. It is interesting to note that cooperativity is observed over a greater range of concentrations compared to pair-wise combinations. It appears that while AmB|PEG-DSPE and rapamycin|PEG-DSPE are largely indifferent, the three-drug combination derives synergistic potency from interactions between 5-FC-AmB|PEG-DSPE and 5-FC-rapamycin|PEG-DSPE. We would like to call attention to the fact that complete inhibition of fungal growth was observed for C. albicans 98-17 and 98-234 for ternary mixtures containing less than 0.03 mg/L AmB|PEG-DSPE, 0.03 mg/L rapamycin|PEG-DSPE and 0.08 mg/L 5-FC.

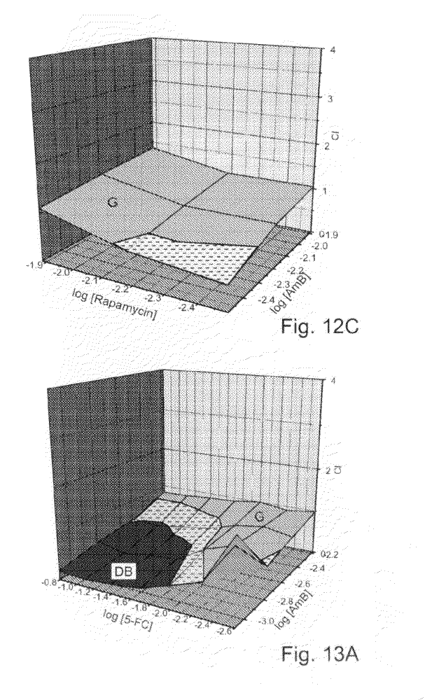

FIG. 12. Contour Plots for 5-FC-AmB|PEG-DSPE b. 5-FC-rapamycin|PEG-DSPE c. rapamycin|PEG-DSPE-AmB|PEG-DSPE combinations against C. albicans 98-17

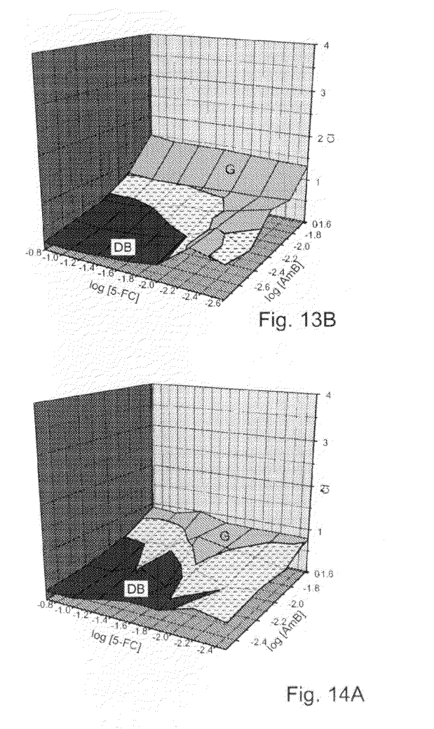

FIG. 13. Contour Plots for 5-FC-AmB|PEG-DSPE-rapamycin|PEG-DSPE combinations for AmB|PEG-DSPE:rapamycin|PEG-DSPE a. 1:2 b. 2:1 for C. albicans 98-17

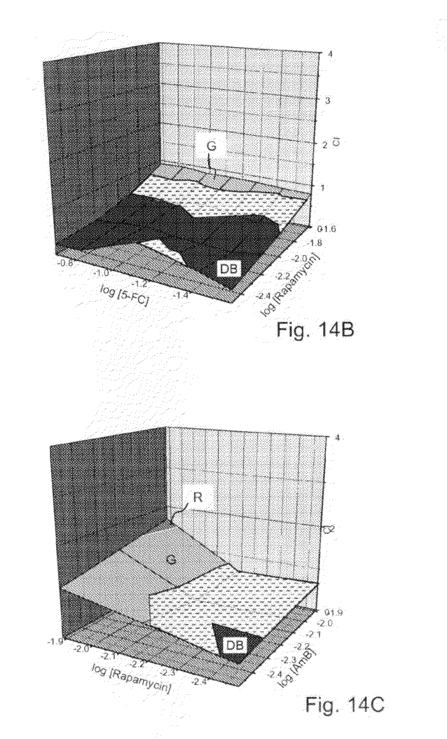

FIG. 14. Contour Plots for a. 5-FC-AmB|PEG-DSPE b. 5-FC-rapamycin|PEG-DSPE c. rapamycin|PEG-DSPE-AmB|PEG-DSPE combinations against C. albicans 98-234

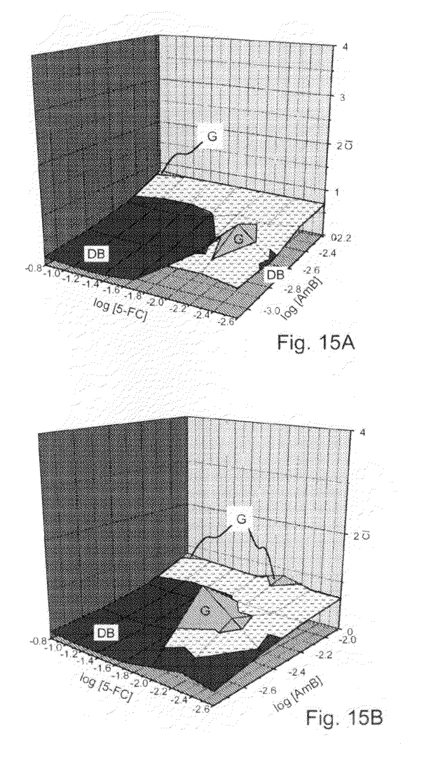

FIG. 15. Contour Plots for 5-FC-AmB|PEG-DSPE-rapamycin|PEG-DSPE combinations for AmB|PEG-DSPE:rapamycin|PEG-DSPE a. 1:2 b. 2:1 for C. albicans 98-234

In summary, there was good agreement between results obtained from FIC analysis and those obtained from the method Chou and Talalay, using the median-effect equation. Each drug pair represented indifferent or moderately synergistic interactions. This analysis points to drug combinations with a high 5-FC: AmB|PEG-DSPE and AmB: rapamycin|PEG-DSPE as being useful for having antifungal effects. In vitro studies can provide an excellent frame-work for studying drug interactions, but factors such as drug pharmacokinetics, fungal burden and state of immunosuppression have been thought to play an important role in outcome in vivo (5). Aspects of embodiments herein are further examined in animal models.

### 8. Conclusions

AmB|PEG-DSPE represents a useful alternative to D-AmB, with significant advantages in the context of combination therapy. In this form, AmB is stable against precipitation in a saline vehicle over prolonged periods of time, which may enable slow or continuous administration of AmB with simultaneous sodium supplementation in a single IV access-line. Contrary to L-AmB, which exerts lower activity compared to D-AmB, AmB|PEG-DSPE retains potent antifungal in vitro efficacy. Additionally, in this form AmB may be mixed with other antifungal agents such 5-FC and rapamycin|PEG-DSPE, allowing further options of combination therapy for the treatment of invasive mycoses in animal models.

In this investigation, we observed indifferent or moderately synergistic activity for pair-wise drug combinations against C. albicans isolates tested. Drug combinations with a high ratio of 5-FC:AmB|PEG-DSPE and 5-FC:rapamycin|PEG-DSPE exhibited cooperative interactions and are of interest since they enable a reduction of 5-FC levels in the presence of low levels of AmB|PEG-DSPE and rapamycin|PEG-DSPE. Additionally, drug combinations may present a broad range activity, particularly in the context of resistant isolates. The studies herein indicate that the three-drug combination was favorable over a wider range of drug ratios compared to pair-wise combinations, presumably due to cooperative 5-FC-AmB|PEG-DSPE and 5-FC-rapamycin|PEG-DSPE interactions.

The embodiments of formulations herein described can provide useful compositions and methods including such, e.g., directed to efficacy against disseminated candidiasis. Embodiments of certain formulations can provide some improvement in toxicity over D-AmB—particularly in conjunction with saline loading and slow administration.

### 9. References for Example 2

1. Gudlaugsson, O., Gillespie, S., Lee, K., Vande Berg, J., Hu, J., Messer, S., Herwaldt, L., Pfaller, M., Diekema, D., Attributable mortality of nosocomial candidemia, revisited. Clin. Infect. Dis., 2003. 37(9): p. 1172-7.
2. Spellberg, B., Filler, S., Edwards, J., Current treatment strategies for disseminated candidiasis. Clin. Infect. Dis., 2006. 42(2): p. 244-51.
3. Dupont, B., Overview of the lipid formulations of amphotericin B. J. Antimicrob. Chemother., 2002. 49 Suppl. 1: p. 31-6.
4. Walsh, T., Finberg, R., Arndt, C., Hiemenz, J., Schwartz, C., Bodensteiner, D., Pappas, P., Seibel, N., Greenberg, R., Dummer, S., Schuster, M., Holcenberg, J., Dismukes, W., Liposomal amphotericin B for empirical therapy in patients with persistent fever and neutropenia. N. Engl. J. Med., 1999. 340(10): p. 764-771.
5. Johnson, M., Macdougall, C., Ostrosky-Zeichner, L., Perfect, J., Rex, J., Combination antifungal therapy. Antimicrob. Agents Chemother., 2004. 48(3): p. 693-715.
6. Mukherjee, P., Sheehan, D., Hitchcock, C., Ghannoum, M., Combination treatment of invasive fungal infections. Clin. Microbiol. Rev., 2005. 18(1): p. 163-94.
7. Kume, H., Murase, S., Mochizuki, M., Combined effect of antifungal agents—fundamental study on amphotericin B and flucytosine. Jpn. J. Med. Mycol., 1985. 26: p. 126-132.
8. Sobel, J., Combination therapy for invasive mycoses: Evaluation of past clinical trial designs. Clin. Infect. Dis., 2004. 39 Suppl. 4: p. S224-7.
9. Arroyo, J., Medoff, G., Kobayashi, G. S., Therapy of murine aspergillosis with amphotericin B in combination with rifampin of 5-fluorocytosine. Antimicrob. Agents Chemother., 1977. 11(1): p. 21-25.
10. Te Dorsthorst, D., Verweij, P., Meletiadis, J., Bergervoet, M., Punt, N., Meis, J., Mouton, J., In vitro interaction of flucytosine combined with amphotericin B or fluconazole against thirty-five yeast isolates determined by both the fractional inhibitory concentration index and the response surface approach. Antimicrob. Agents Chemother., 2002. 46(9): p. 2982-9.
11. Medoff, G., Kobayashi, G., Kwan, C., Schlessinger, D., Venkov, P., Potentiation of rifampicin and 5-fluorocytosine as antifungal antibiotics by amphotericin B (yeast-membrane permeability-ribosomal RNA-eukaryotic cell-synergism). Proc. Natl. Acad. Sci., 1972. 69(1): p. 196-9.
12. Hope, W., Warn, P., Sharp, A., Reed, P., Keevil, B., Louie, A., Denning, D., Drusano, G., Surface response modeling to examine the combination of amphotericin b deoxycholate and 5-fluorocytosine for treatment of invasive candidiasis. J. Infect. Dis., 2005. 192: p. 673-680.
13. Vezina, C., Kudelski, A., Sehgal, S., Rapamycin (AY-22,989), a new antifungal antibiotic I. Taxonomy of the producing streptomycete and isolation of the active principle. J. of Antibiotics, 1975. 28: p. 721-726.
14. Baker, H., Sidorowicz, A., Sehgal, S., Vezina, C., Rapamycin (AY-22,989), a new antifungal antibiotic III. In vitro and in vivo evaluation. J. Antibiotics, 1978. 31: p. 539-545.
15. Kahan, B., Camardo, J., Rapamycin: clinical results and future opportunities. Transplantation, 2001. 72: p. 1181-1193.
16. Dickman, D., Ding, H., Li, Q., Nilius, A., Balli, D., Ballaron, S., Trevillyan, J., Smith, M., Seif, L., Kim, L., Sarthy, A., Goldman, R., Plattner, J., Bennani Y., Antifungal rapamycin analogues with reduced immunosuppressive activity. Bioorg. Med. Chem. Lett., 2000. 10: p. 1405-1408.
17. Rohde, J., Heitman, J., Cardenas, M., The TOR kinases link nutrient sensing to cell growth. J. Biol. Chem., 2001. 276(13): p. 9583-6.
18. Imhof, A., Walter, R. B., Schaffner, A., Continuous infusion of escalated doses of amphotericin b deoxycholate: an open-label observational study. Clin. Infect. Dis., 2003. 36: p. 943-951.
19. Eriksson, U., Seifer, B., Schaffner, A., Comparison of effects of amphotericin b deoxycholate infused over 4 to 24 hours: Randomized controlled trial. Br. Med. J., 2001. 322: p. 579-582.
20. Peleg, A., Woods, M., Continuous and 4 h infusion of amphotericin B: a comparative study involving high-risk haematology patients. J. Antimicrob. Chemother., 2004. 54(4): p. 803-8.
21. Lewis, R., Wiederhold, N., The solubility ceiling: a rationale for continuous infusion of amphotericin b therapy? Clin. Infect. Dis., 2003. 37: p. 871-872.
22. Llanos, A., Cieza, J., Berando, J., Effect of salt supplementation on amphotericin nephrotoxicity. Kidney Int., 1991. 40: p. 302-308.
23. Jurgens, R., Deluca, P., Papadimitriou D., Compatibility of amphotericin b with certain large-volume parenterals. Am. J. Hospital Pharm., 1981. 38: p. 377-378.
24. Vakil, R., Kwon, G., PEG-phospholipid micelles for the delivery of amphotericin B. J. Controlled Release, 2005. 101(1-3): p. 386-9.
25. Pfaller, M., Reference method for broth dilution antifungal susceptibility testing of yeasts: approved standard. 2002 ed. NCCLS, M27-A2. Vol. 22 no. 15. 2002, Wayne, Pa.: National Committee for Clinical Laboratory Standards.
26. Chou, T.-C., Talalay, P., Quantitative analysis of dose-effect relationships: the combined effects of multiple drugs or enzyme inhibitors. Adv.

Enzyme Regul., 1984. 22: p. 27-55.
27. Chou, T.-C., Talalay, P., Generalized equations for the analysis of inhibitions of Michaelis-Menten and higher-order kinetic systems with two or more mutually exclusive and nonexclusive inhibitors. Eur. J. Biochem., 1981. 115(1): p. 207-16.
28. Instructions to Authors. Antimicrob. Agents Chemother., 2006. 50(1): p. 1-21.
29. Odds, F., Synergy, antagonism, and what the chequerboard puts between them. J. Antimicrob. Chemother., 2003. 52(1): p. 1.
30. Chou, T.-C., Theoretical basis, experimental design, and computerized simulation of synergism and antagonism in drug combination studies. Pharmacol. Rev., 2006. 58(3): p. 621-681.
31. Espuelas, M., Legrand, P., Campanero, M., Appel, M., Cheron, M., Gamazo, C., Barratt, G., Irache, J., Polymeric carriers for amphotericin B: In vitro activity, toxicity and therapeutic efficacy against systemic candidiasis in neutropenic mice. J. Antimicrob. Chemother., 2003. 52(3): p. 419-27.
32. Nakagawa, Z., Nucci, M., Branchini, M., Salomao, R., Richtmann, R., Colombo, A. In vitro susceptibility patterns of 200 recent bloodstream isolates of Candida species to four antifungal drugs determined by the NCCLS microbroth procedure. in Intersci. Conf. Antimicrob. Agents Chemother. 1998.
33. Ghannoum, M., Motawy, M., Abu Hatab, M., Ibrahim, A., Griddle, R., Multifactorial analysis of effects of interactions among antifungal and antineoplastic drugs on inhibition of Candida albicans growth. Antimicrob. Agents Chemother., 1989. 33(5): p. 717-25.
34. Greco, W., Bravo, G., Parsons, J., The search for synergy: a critical review from a response surface perspective. Pharmacol. Rev., 1995. 47(2): p. 331-85.
35. Greco, W., Park, H., Rustum, Y., Application of a new approach for the quantitation of drug synergism to the combination of cis-diamminedichloroplatinum and 1-beta-D-arabinofuranosylcytosine. Cancer Res., 1990. 50(17): p. 5318-27.
36. Steinbach, W., Schell, W., Blankenship, J., Onyewu, C., Heitman, J., Perfect, J., In vitro interactions between antifungals and immunosuppressants against Aspergillus fumigatus. Antimicrob. Agents Chemother., 2004. 48(5): p. 1664-9.
37. Shadomy, S., Wagner, G., Espinel-Ingroff, E., Davis, B. A., In vitro studies with combinations of 5-fluorocytosine and amphotericin B. Antimicrob. Agents Chemother., 1975. 8(2): p. 117-21.
38. Ghannoum, M., Motawy, M., Abu Hatab, M., Abu Elteen, K., Griddle, R., Interactive effects of antifungal and antineoplastic agents on yeasts commonly prevalent in cancer patients. Antimicrob. Agents Chemother., 1989. 33(5): p. 726-30.

### STATEMENTS REGARDING INCORPORATION BY REFERENCE AND VARIATIONS

All references mentioned throughout this application, for example patent documents including issued or granted patents or equivalents; patent application publications; unpublished patent applications; and non-patent literature documents or other source material; are hereby incorporated by reference herein in their entireties, as though individually incorporated by reference. In the event of any inconsistency between cited references and the disclosure of the present application, the disclosure herein takes precedence. Some references provided herein are incorporated by reference to provide information, e.g., details concerning sources of starting materials, additional starting materials, additional reagents, additional methods of synthesis, additional methods of analysis, additional biological materials, additional cells, and additional uses of the invention.

The following references relate generally to Rapamycin analogs and are incorporated by reference to the extent not inconsistent with the disclosure herein: (1) Rapamycin and Less Immunosuppressive Analogs Are Toxic to Candida albicans and Cryptococcus neoformans via FKBP12-Dependent Inhibition of TOR. Antimicrobial Agents And Chemotherapy, 45(11) p 3162-3170 (2001); and (2) Dickman, D. A. et al. (2000) Antifungal rapamycin analogues with reduced immunosuppressive activity. Bioorg. Med. Chem. Lett. 10:p 1405-1408. The following references relate generally to Rapamycin prodrugs and are incorporated by reference to the extent not inconsistent with the disclosure herein: (1) G. Nudes, M. Carducci, P. Tomczak, J. Dutcher, R. Figlin, A. Kapoor, E. Staroslawska, T. O'Toole, Y. Park, L. Moore. A phase 3, randomized, 3-arm study of temsirolimus (TEMSR) or interferon-alpha (IFN) or the combination of TEMSR+IFN in the treatment of first-line, poor-risk patients with advanced renal cell carcinoma (adv RCC). (2) Phase I/II Study of the Mammalian Target of Rapamycin Inhibitor Everolimus (RAD001) in Patients with Relapsed or Refractory Hematologic Malignancies. Karen W. L. Yee et al. Clinical Cancer Research Vol. 12, 5165-5173, Sep. 1, 2006; (3) Phase I Trial of the Novel Mammalian Target of Rapamycin Inhibitor Deforolimus (AP23573; MK-8669) Administered Intravenously Daily for 5 Days Every 2 Weeks to Patients With Advanced Malignancies. Monica M. Mita et al. Journal of Clinical Oncology, Vol 26, No 3 (January 20), 2008: pp. 361-367; and (4) Survival results with AP23573, a novel mTOR inhibitor, in patients (pts) with advanced soft tissue or bone sarcomas: Update of phase II trial. S. P. Chawla et al. 2007 ASCO Annual Meeting. Abstract 10076.

All patents and publications mentioned herein are indicative of the levels of skill of those skilled in the art to which the invention pertains. References cited herein can indicate the state of the art as of their publication or filing date, and it is intended that this information can be employed herein, if needed, to exclude specific embodiments that are in the prior art. For example, when composition of matter are claimed herein, it should be understood that compounds known and available in the art prior to Applicant's invention, including compounds for which an enabling disclosure is provided in the references cited herein, are not intended to be included in the composition of matter claims herein.

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Thus as used herein, comprising is synonymous with including, containing, having, or characterized by, and is inclusive or open-ended and does not exclude additional, unrecited elements or method steps. As used herein, “consisting of” excludes any element, step, or ingredient, etc. not specified in the claim description. As used herein, “consisting essentially of” does not exclude materials or steps that do not materially affect the basic and novel characteristics of the claim (e.g., relating to an active ingredient). In each instance herein any of the terms “comprising”, “consisting essentially of” and “consisting of” may be replaced with at least either of the other two terms, thereby disclosing separate embodiments and/or scopes which are not necessarily coextensive. An embodiment of the invention illustratively described herein suitably may be practiced in the absence of any element or elements or limitation or limitations not specifically disclosed herein.

Whenever a range is disclosed herein, e.g., a temperature range, time range, composition or concentration range, or other value range, etc., all intermediate ranges and subranges as well as all individual values included in the ranges given are intended to be included in the disclosure. This invention is not to be limited by the embodiments disclosed, including any shown in the drawings or exemplified in the specification, which are given by way of example or illustration and not of limitation. It will be understood that any subranges or individual values in a range or subrange that are included in the description herein can be excluded from the claims herein.

The invention has been described with reference to various specific and/or preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be employed in the practice of the invention as broadly disclosed herein without resort to undue experimentation; this can extend, for example, to starting materials, biological materials, reagents, synthetic methods, purification methods, analytical methods, assay methods, and biological methods other than those specifically exemplified. All art-known functional equivalents of the foregoing (e.g., compositions, methods, devices, device elements, materials, procedures and techniques, etc.) described herein are intended to be encompassed by this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, preferred embodiments, and optional features, modification and variation of the concepts herein disclosed may be resorted to by those skilled in the art, and that such modifications and variations are considered to be within the scope of this invention as defined by the appended claims.

### REFERENCES

U.S. Pat. No. 6,168,804 by John Samuel and Glen Kwon, Method for eliciting Th1-specific immune response, issued Jan. 2, 2001. U.S. Pat. No. 6,413,537 by Glen S. Kwon, et al., Nystatin formulation having reduced toxicity, issued Jul. 2, 2002. U.S. Pat. No. 6,939,561 by Glen S. Kwon, et al., Methods and compositions for polyene antibiotics with reduced toxicity, issued Sep. 6, 2005.
This application incorporates by reference, to the extent not inconsistent with the disclosure herewith, the following applications: U.S. application Ser. 10/404,926 filed Mar. 31, 2003 (published as 20040005351 on Jan. 8, 2004); and U.S. Ser. 10/687,558 filed Oct. 15, 2003 (published as 20040116360 on Jun. 17, 2004).
U.S. Pat. No. 6,217,886 by Onyuksel, Hayat, and Rubinstein, Israel for Materials and methods for making improved micelle compositions; and U.S. Pat. No. 6,322,810 by Onyuksel et al. for Materials and methods for making improved micelle compositions; U.S. Pat. No. 6,348,214 for Materials and methods for making improved liposome compositions;
U.S. Pat. No. 6,197,333 for Materials and methods for making improved liposome compositions.
U.S. Pat. Nos. 6,043,094, 6,143,321, 5,891,468, 6,897,196, 7,179,484
U.S. Pat. No. 6,897,196 by Szoka, Jr., et al. May 24, 2005, pH sensitive lipids based on ortho ester linkers, composition and method.
U.S. Pat. No. 5,827,533 Needham Oct. 27, 1998 Liposomes containing active agents aggregated with lipid surfactants;
U.S. Pat. No. 6,660,525 Martin, et al. Dec. 9, 2003 Therapeutic liposome composition and method; U.S. Pat. No. 6,936,272
U.S. Pat. No. 6,316,024 Allen, et al. Nov. 13, 2001 Therapeutic liposome composition and method of preparation
U.S. Pat. No. 7,105,229 Anderson Sep. 12, 2006 Coated particles, methods of making and using
U.S. Pat. No. 7,125,546 Kataoka, et al. Oct. 24, 2006 Polymeric micelle containing cisplatin enclosed therein and use thereof.
U.S. Pat. No. 5,444,052; U.S. Pat. No. 7,122,202
References relating in part to Amphotec® complex of amphotericin B and cholesteryl sulfate: U.S. Pat. Nos. 4,822,777; 5,032,582; 5,194,266; 5,077,057.
US 20030086964A1 by Glen S. Kwon et al., Methods and compositions for polyene antibiotics with reduced toxicity, published May 8, 2003.
US 20040005351A1 by Glen S. Kwon et al., Polymeric micelle formulations of hydrophobic compounds and methods, published Jan. 8, 2004.
US 20040116360 by Glen S. Kwon, et al., Encapsulation and deaggregation of polyene antibiotics using poly(ethylene glycol)-phospholipid micelles, published Jun. 17, 2004.
US 20060251710 by Glen S. Kwon, et al., Micelle composition of polymer and passenger drug, published Nov. 9, 2006.
20060105968 A1 Cleary; John D.; et al. May 18, 2006
Asher I M, 1977, Biophysical Journal vol. 17, 1977 at 49a. W-PM-C8. Effects of temperature and molecular interactions on the vibrational infrared spectra of phospholipid vesicles.
Baas B, 1999, AAPS Pharmsci 1999; 1 (3) article 10 (http://www.pharmsci.org) Activity and Kinetics of Dissociation and Transfer of Amphotericin B from a Novel Delivery Form.
Barwicz J, Tancrede P. Chem Phys Lipids. 1997 Feb. 28; 85(2):145-55. The effect of aggregation state of amphotericin-B on its interactions with cholesterol- or ergosterol-containing phosphatidylcholine monolayers.
Boswell G W, ANTIMICROBIAL AGENTS AND CHEMOTHERAPY, February 1998, p. 263-268
Croy S R, Kwon G S, Curr Pharm Des. 2006; 12(36):4669-84. Polymeric micelles for drug delivery.
Doubek M et al., Nephrology Volume 10 Issue 1 Page 57-February 2005, Comparison of the effect of amphotericin B desoxycholate and amphotericin B colloidal dispersion on renal functions and renal morphology in rats
Falk R, ANTIMICROBIAL AGENTS AND CHEMOTHERAPY, August 1999, p. 1975-1981
Golenser J, Mini Rev Med. Chem. 2006 February; 6(2):153-62. New formulations and derivatives of amphotericin B for treatment of leishmaniasis.
Johnson, Melissa D. et al., Antimicrobial Agents and Chemotherapy, March 2004, p. 693-715, Vol. 48, No. 3, Combination Antifungal Therapy.
Johnsson, Markus, and Katarina Edwards, Biophys J. 2003 December; 85(6): 3839-3847. Liposomes, Disks, and Spherical Micelles: Aggregate Structure in Mixtures of Gel Phase Phosphatidylcholines and Poly(Ethylene Glycol)-Phospholipids.
Kawabata, Manami, J. Biochem. Vol. 129, pp. 725-732 (2001), Effect of Aggregation of Amphotericin B on Lysophosphatidylcholine Micelles as Related to Its Complex Formation with Cholesterol or Ergosterol.
Kotler-Brajtburg J, ANTIMICROBIAL AGENTS AND CHEMOTHERAPY, May 1979, p. 716-722 Vol. 15, No. 5, Classification of Polyene Antibiotics According to Chemical Structure and Biological Effects.
Larabi M, Study of the toxicity of a new lipid complex formulation of amphotericin B, Journal of Antimicrobial Chemotherapy (2004) 53, 81-88
Meletiadis Joseph, Clinical Microbiology Reviews, October 2006, p. 763-787, Vol. 19, No. 4, Human Pharmacogenomic Variations and Their Implications for Antifungal Efficacy
Moribe K et al., Pharm Res. 1998 November; 15(11):1737-42. Enhanced encapsulation of amphotericin B into liposomes by complex formation with polyethylene glycol derivatives. Moribe K, Tanaka E, Maruyama K, Iwatsuru M.
Moribe K, Maruyama K, Iwatsuru M., Int J. Pharm. 1999 Dec. 20; 193(1):97-106. Molecular localization and state of amphotericin B in PEG liposomes.
Moribe K., International journal of pharmaceutics (Int. j. pharm.), 1999, vol. 188, not, pp. 193-202, Encapsulation characteristics of nystatin in liposomes: effects of cholesterol and polyethylene glycol derivatives.
Moribe, Kunikazu and Kazuo Maruyama, Current Pharmaceutical Design, 2002, 8(6):441-454, Pharmaceutical Design of the Liposomal Antimicrobial Agents for Infectious Disease.
Ng, Agatha W. K., Kishor M. Wasan, Pharm Pharmaceut Sci (www.ualberta.ca/˜csps) 6(1):67-83, 2003, Development of liposomal polyene antibiotics: an historical perspective.
Oda M N et al., Journal of Lipid Research, Vol. 47, 260-267, February 2006, Reconstituted high density lipoprotein enriched with the polyene antibiotic amphotericin B.
Onda M, J Biochem (Tokyo). 2003 July; 134(1):121-8. Susceptibilities of phospholipid vesicles containing different sterols to amphotericin B-loaded lysophosphatidylcholine micelles.
Onda, Maki, J. Biochem, 2003, Vol. 134, No. 1 121-128, Japanese Biochemical Society, Susceptibilities of Phospholipid Vesicles Containing Different Sterols to Amphotericin B-Loaded Lysophosphatidylcholine Micelles.
Onyewu, Chiatogu et al., Anti-Infective Agents in Medicinal Chemistry, 2007, 6, 3-15, Unique Applications of Novel Antifungal Drug Combinations.
Risovic, V, ANTIMICROBIAL AGENTS AND CHEMOTHERAPY, October 2003, p. 3339-3342 Vol. 47, No. 10
Robinson R F PharmD, M. C. Nahata PharmD (1999) A comparative review of conventional and lipid formulations of amphotericin B, Journal of Clinical Pharmacy and Therapeutics 24 (4), 249-257.
Vandermeulen G, International Journal of Pharmaceutics 309 (2006) 234-240, Pharmaceutical Nanotechnology, Encapsulation of amphotericin B in poly(ethylene glycol)-block-poly(c-caprolactone-co-trimethylenecarbonate) polymeric micelles.
Yoo B K, 2006, Biol Pharm Bull. 2006 August; 29(8):1700-5. Reduced renal toxicity of nanoparticular amphotericin B micelles prepared with partially benzylated poly-L-aspartic acid.
Vakil R, Kwon G S, Langmuir. 2006 Nov. 7; 22(23):9723-9, Poly(ethylene glycol)-b-poly(epsilon-caprolactone) and PEG-phospholipid form stable mixed micelles in aqueous media.
Vakil R and Kwon, Glen S., Mol. Pharmaceutics, 5 (1), 98-104, 2008. Effect of Cholesterol on the Release of Amphotericin B from PEG-Phospholipid Micelles. Epub 2007 December 27
Vakil R, Knilans K, Andes D, Kwon GS, 2008, Pharm Res., 2008 April 16, Combination Antifungal Therapy Involving Amphotericin B, Rapamycin and 5-Fluorocytosine Using PEG-Phospholipid Micelles.
Clemons K V, Stevens D A, Antimicrob Agents Chemother. 1998 April; 42(4): 899-902. PMCID: PMC105563, Comparison of Fungizone, Amphotec, AmBisome, and Abelcet for Treatment of Systemic Murine Cryptococcosis.
Clemons K V, Parmar R, Martinez M, Stevens D A, Efficacy of Abelcet alone, or in combination therapy, against experimental central nervous system aspergillosis, J Antimicrob Chemother. 2006 August; 58(2):466-9. Epub 2006 June 7
Clemons K V, Espiritu M, Parmar R, Stevens D A. Comparative efficacies of conventional amphotericin b, liposomal amphotericin B (AmBisome), caspofungin, micafungin, and voriconazole alone and in combination against experimental murine central nervous system aspergillosis. Antimicrob Agents Chemother. 2005 December; 49(12):4867-75.
Clemons K V, Stevens D A. Comparative efficacies of four amphotericin B formulations—Fungizone, amphotec (Amphocil), AmBisome, and Abelcet—against systemic murine aspergillosis. Antimicrob Agents Chemother. 2004 March; 48(3):1047-50.
Van Etten, Els W M et al., Antimicrobial Agents and Chemotherapy, 1995, p. 1954-1958 Vol. 39, No. 9, Amphotericin B Liposomes with Prolonged Circulation in Blood: In Vitro Antifungal Activity, Toxicity, and Efficacy in Systemic Candidiasis in Leukopenic Mice.
Van Etten, Els W M et al., Sterically stabilized amphotericin B-liposomes: toxicity and biodistribution in mice, Journal of Controlled Release, Volume 37, Issues 1-2, November 1995, Pages 123-129.
Vakil R., Kuldipkumar A., Andes D., Tan Y., Kwon G., “Polymeric Micelles for the Delivery of Polyene Antibiotics”, in S. Svenson (Ed.), Polymeric Drug Delivery Vol. 1—Particulate Drug Carriers, ACS Symposium Series Vol. 923, New York (2006), 14-26.

We claim:

1. A combination antifungal composition comprising an Amphotericin B component and at least a second antifungal agent, wherein said Amphotericin B and said second antifungal agent are solubilized in an aqueous salt solution.

2. The combination antifungal composition of claim 1 wherein said Amphotericin B component comprises AmB:PEG-DSPE.

3. The combination antifungal composition of claim 1 or 2 wherein said second antifungal agent comprises a rapamycin component.

4. The combination antifungal composition of claim 3 wherein said rapamycin component comprises rapamycin:PEG-DSPE.

5. The combination antifungal composition of any of claims 1-4 wherein said second antifungal agent comprises 5-fluorocytosine.

6. The combination antifungal composition of any of claims 1-4 wherein said second antifungal agent comprises a rapamycin analog selected from the group consisting of: (S)—NOHCON-Piperidyl-Rapamycin; 1,2,3,4-tetrahydro-rapamycin; (S)—NOHCOOiBu-Rapamycin; (S)-2-Me-Thienyl-Rapamycin; (S)—OH-Rapamycin; and Desmethoxyrapamycin.

7. The combination antifungal composition of any of claims 1-4 wherein said second antifungal agent comprises a rapamycin prodrug selected from the group consisting of: 42-[3-Hydroxy-2-(hydroxymethyl)-2-methylpropanoate]rapamycin (Temsirolimus/CCI-779); 42-O-(2-Hydroxy)ethyl rapamycin (Everolimus/RAD001); Deforolimus/MK-8669; 42-(Dimethylphosphinate)rapamycin; Mono-(28)-N,N-dimethylglycinate-rapamycin; Mono-(28)-4-(pyrrolidino)butyrate-rapamycin; and Mono-(28)-N,N-diethylpropionate rapamycin.

8. The combination antifungal composition of any of claims 1-7 further comprising a third antifungal agent.

9. The combination antifungal composition of any of claims 1-8 wherein at least one of said Amphotericin B component and said second antifungal agent is in a mixture comprising PEG-DSPE.

10. The combination antifungal composition of any of claims 1-9 wherein at least one of said Amphotericin B component and said second antifungal agent is in a substantially micellar phase.

11. The combination antifungal composition of any of claims 1-10 wherein said Amphotericin B component and said second antifungal agent are in a co-formulated mixture comprising PEG-DSPE.

12. The combination antifungal composition of any of claims 8-11 wherein said Amphotericin B component comprises AmB:PEG-DSPE, said second antifungal agent comprises rapamycin:PEG-DSPE, and said third antifungal agent comprises 5-fluorocytosine.

13. A method of inhibiting a fungal agent, comprising: contacting said fungal agent with the composition of any of claims 1-12.

14. A method of treating a fungal infection, comprising administering to a subject in need thereof an effective amount of the composition of any of claims 1-12, thereby treating the fungal infection.

15. A composition of PEG-PL:STX:RX for delivery of a pharmaceutical agent, comprising a poly(ethylene glycol)-phospholipid (PEG-PL), a sterol (STX), and the pharmaceutical agent (RX), wherein said RX is provided in a deaggregated and substantially micellar phase.

16. The composition of claim 15 wherein said phospholipid is 1,2-Distearoyl-sn-glycero-3-phosphoethanolamine (DSPE) and thereby said PEG-phospholipid is PEG-DSPE, thus PEG-DSPE:STX:RX.

17. The composition of claim 15 or 16 wherein said sterol is cholesterol (CHOL), thus PEG-DSPE:CHOL:RX.

18. The composition of any of claims 15-17 wherein said pharmaceutical agent is a polyene antibiotic.

19. The composition of any of claims 15-18 wherein said pharmaceutical agent is Amphotericin B (AmB).

20. The composition of any of claims 15-17 wherein said pharmaceutical agent is selected from the group consisting of Amphotericin B (AmB) and rapamycin.

21. The composition of any of claims 15-16 wherein said phospholipid is DSPE, said sterol is cholesterol (CHOL), and said pharmaceutical agent is Amphotericin B; thus PEG-DSPE:CHOL:AmB.

22. The composition of any of claims 15-21 wherein said micellar phase comprises micelles having an average diameter of less than about 100 nanometers.

23. The composition of any of claims 15-22 wherein said micellar phase comprises micelles having an average diameter of less than about 100 nanometers and greater than about 10 nanometers.

24. The composition of any of claims 15-16, wherein said sterol is selected from the group consisting of: cholesterol, ergosterol, lanosterol, beta-sitosterol or stigmasterol.

25. The composition of claim 15 wherein said pharmaceutical agent is hydrophobic.

26. The composition of claim 15 further comprising an aqueous salt solution.

27. The composition of claim 26 wherein said aqueous salt solution comprises aqueous sodium chloride (NaCl).

28. A composition of particles comprising AmB, PEG-DSPE, and CHOL having a ratio of from about 1:2:0.25 to about 1:2:2 for AmB:PEG-DSPE:CHOL.

29. A pharmaceutical formulation of a pharmaceutical agent, comprising the composition according to any of claims 1-12 and 15-28 and a pharmaceutical carrier and/or excipient.

30. A method for delivery of a pharmaceutical agent to a subject, comprising providing a composition or formulation according to any of claims 1-12 and 15-28, and administering said composition or formulation to said subject, thereby achieving delivery of said pharmaceutical agent to said subject.

31. The method of claim 30, wherein the sterol is capable of reducing a first release rate of said pharmaceutical agent in said PEG-PL:STX:RX composition relative to a second release rate of the pharmaceutical agent in a PEG-PL:RX composition.

32. The method of claim 31, wherein said first release rate and said second release rate are independently measured in a proteinaceous fluid.

33. A combination antifungal composition comprising the composition of any of claims 15-17 and a second pharmaceutical agent, wherein at least one of said pharmaceutical agent and said second pharmaceutical agent is an antifungal agent.

34. The combination composition of claim 33 wherein said antifungal agent is Amphotericin B.

35. The combination composition of claim 33 wherein said antifungal agent is fluconazole.

36. The combination composition of claim 33 wherein said antifungal agent is 5-fluorocytosine.

37. The combination composition of claim 33 wherein said pharmaceutical agent is Amphotericin B and said second pharmaceutical agent is fluconazole or 5-fluorocytosine.

38. The combination composition of claim 33 wherein at least one of said pharmaceutical agent and said second pharmaceutical agent is selected from the group consisting of Amphotericin B, rapamycin, nystatin, 5-fluorocytosine; fluconazole (also known as 2-(2,4-difluorophenyl)-1,3-bis(1H-1,2,4-triazol-1-yl)propan-2-ol) (S)—NOHCON-Piperidyl-Rapamycin; 1,2,3,4-tetrahydro-rapamycin; (S)—NOHCOOiBu-Rapamycin; (S)-2-Me-Thienyl-Rapamycin; (S)—OH-Rapamycin; Desmethoxyrapamycin; 42-[3-Hydroxy-2-(hydroxymethyl)-2-methylpropanoate]rapamycin (Temsirolimus/CCI-779); 42-O-(2-Hydroxy)ethyl rapamycin (Everolimus/RAD001); Deforolimus/MK-8669; 42-(Dimethylphosphinate)rapamycin; Mono-(28)-N,N-dimethylglycinate-rapamycin; Mono-(28)-4-(pyrrolidino)butyrate-rapamycin; and Mono-(28)-N,N-diethylpropionate rapamycin.

39. A method of co-administration of a plurality of pharmaceutical agents to a subject in need thereof, comprising:
(a) providing a micelle composition of a first pharmaceutical agent according to any of claims 1-10 and 13-26, wherein said micelle composition is compatible with an aqueous NaCl solution for sodium supplementation to said subject;
(b) providing at least a second pharmaceutical agent;
(c) mixing said first and second pharmaceutical agents, thereby generating a mixture; and
(d) administering said mixture to said subject;
thereby achieving co-administration of said plurality of pharmaceutical agents.

40. The method of claim 39 wherein at least one of said plurality of pharmaceutical agents is an antifungal agent.

41. The method of claim 39 wherein said first pharmaceutical agent comprises Amphotericin B.

42. The method of claim 39 wherein each of said first and second pharmaceutical agents is an antifungal agent.

43. The method of claim 39 further comprising the step (e) administering a sodium supplementation to said subject, wherein the administering of said sodium is before, concurrent with, or after step (d).

44. The method of any of claims 39-43 wherein said second pharmaceutical agent is rapamycin, 5-fluorocytosine, or fluconazole.

45. A method of making an AmB composition, wherein said AmB composition is in a substantially deaggregated form and capable of being soluble in an aqueous salt solution, comprising providing AmB, providing PEG-DSPE, and mixing said AmB with said PEG-DSPE; thereby generating said AmB composition.

46. A method of making an AmB composition, wherein said AmB composition is in a substantially deaggregated form and capable of being soluble in an aqueous salt solution, comprising providing AmB, providing PEG-DSPE, providing CHOL, dissolving said AmB, PEG-DSPE, and CHOL in a solvent mixture comprising methanol and chloroform, evaporating said solvent to allow dissolution of a thin film sample, equilibrating said sample at a temperature of 50 degrees centigrade, and filtering said sample using an 0.2 micron filter of polyethersulfone; thereby generating said AmB composition.

47. The method of claim 45 or 46 wherein it is not necessary to formulate said AmB composition with sodium deoxycholate.

48. The method of any of claims 45-47 wherein said AmB composition is formulated in the absence of a substantial amount of deoxycholate or is formulated without deoxycholate.

49. A method of preparing the composition of any of claims 1-12, 15-28, and 33-38.

50. A method of preparing the pharmaceutical formulation of claim 29.

51. A micelle composition of PEG-PL:RX comprising a poly(ethylene glycol)-phospholipid (PEG-PL), and a pharmaceutical agent (RX).

52. The micelle composition of claim 51 wherein PEG-PL:RX is PEG-DSPE:RX.

53. A micelle composition of PEG-PL:STX:RX, comprising a poly(ethylene glycol)-phospholipid (PEG-PL), a sterol (STX), and a pharmaceutical agent (RX).

54. A method of inhibiting a fungal agent, comprising: contacting said fungal agent with the composition of any of claims 1-12, 15-28, and 33-38.

55. A method of treating a fungal infection, comprising administering an effective amount of the composition of any of claims 1-12, 15-28, and 33-38 to a subject in need thereof, thereby treating the fungal infection.
